SEQUENCE LISTING (43 sequences)

SEQ ID NO: 1 - protein, 5 aa, Homo sapiens
HKGAL

SEQ ID NO: 2 - protein, 5 aa, Homo sapiens
HEDAI

SEQ ID NO: 3 - protein, 6 aa, Artificial Sequence
  59R1Heavy chain CDR1
SSSGMS

SEQ ID NO: 4 - protein, 17 aa, Artificial Sequence
  59R1Heavy chain CDR2
VIASSGSNTYYADSVKG

SEQ ID NO: 5 - protein, 6 aa, Artificial Sequence
  59R1Heavy chain CDR3
GIFFAI

SEQ ID NO: 6 - protein, 12 aa, Artificial Sequence
  59R1 Light chain CDR1
RASQSVRSNYLA

SEQ ID NO: 7 - protein, 7 aa, Artificial Sequence
  59R1 Light chain CDR2
GASSRAT

SEQ ID NO: 8 - protein, 8 aa, Artificial Sequence
  59R1 Light chain CDR3
QQYSNFPI

SEQ ID NO: 9 - protein, 6 aa, Artificial Sequence
  59R5 Heavy chain CDR3
SIFYTT

SEQ ID NO: 10 - protein, 6 aa, Artificial Sequence
  Heavy chain CDR3 consensus sequence
XXFXXX

SEQ ID NO: 11 - protein, 6 aa, Artificial Sequence
  Alternative heavy chain CDR3
SIFYPT

SEQ ID NO: 12 - protein, 6 aa, Artificial Sequence
  Alternative heavy chain CDR3
SSFFAS

SEQ ID NO: 13 - protein, 6 aa, Artificial Sequence
  Alternative heavy chain CDR3
SSFYAS

SEQ ID NO: 14 - protein, 6 aa, Artificial Sequence
  Alternative heavy chain CDR3
SSFFAT

SEQ ID NO: 15 - protein, 6 aa, Artificial Sequence
  Alternative heavy chain CDR3
SIFYPS

SEQ ID NO: 16 - protein, 6 aa, Artificial Sequence
  Alternative heavy chain CDR3
SSFFAN

SEQ ID NO: 17 - protein, 118 aa, Artificial Sequence
  59R5 Heavy chain variable region
EVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYTTWGQGTLVTVSSAST

SEQ ID NO: 18 - protein, 116 aa, Artificial Sequence
  59R1 Heavy chain VH of 59R1 IgG antibody
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARGIFFAIWGQGTLVTVSSA

SEQ ID NO: 19 - protein, 135 aa, Artificial Sequence
  59R1 heavy chain VH plus mammalian signal sequence
MKHLWFFLLLVAAPRWVLSQVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARGIFFAIWGQGTLVTVSSA

SEQ ID NO: 20 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYPTWGQGTLVTVSSA

SEQ ID NO: 21 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSSFFASWGQGTLVTVSSA

SEQ ID NO: 22 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSSFYASWGQGTLVTVSSA

SEQ ID NO: 23 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSSFFATWGQGTLVTVSSA

SEQ ID NO: 24 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYPSWGQGTLVTVSSA

SEQ ID NO: 25 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSSFFANWGQGTLVTVSSA

SEQ ID NO: 26 - protein, 116 aa, Artificial Sequence
  59R1 Heavy chain VH of 59RGV antibody (germlined variant of 59R1)
EVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARGIFFAIWGQGTLVTVSSA

SEQ ID NO: 27 - protein, 110 aa, Artificial Sequence
  59R1 Light chain VL of 59RGV antibody (germlined variant of 59R1)
EIVLTQSPATLSLSPGERATLSCRRASQSVRSNYLAWYQQKPGQAPRLLIYGASSRATGIPARFSGSGSGTDFTLTISSLEPEDFAVYYCQQYSNFPITFGQGTKVEIKR

SEQ ID NO: 28 - protein, 129 aa, Artificial Sequence
  59R1 light chain VL plus mammalian signal sequence
MVLQTQVFISLLLWISGAYGDIVLTQSPATLSLSPGERATLSCRASQSVRSNYLAWYQQKPGQAPRLLIYGASSRATGVPARFSGSGSGTDFTLTISSLEPEDFAVYYCQQYSNFPITFGQGTKVEIKR

SEQ ID NO: 29 - protein, 109 aa, Artificial Sequence
  59R1 Light chain VL of 59R1 IgG antibody
DIVLTQSPATLSLSPGERATLSCRASQSVRSNYLAWYQQKPGQAPRLLIYGASSRATGVPARFSGSGSGTDFTLTISSLEPEDFAVYYCQQYSNFPITFGQGTKVEIKR

SEQ ID NO: 30 - protein, 441 aa, Artificial Sequence
  59R5 Heavy chain
EVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYTTWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 460 aa, Artificial Sequence
  Predicted protein sequence of anti-NOTCH2/3 59R1 IgG2 heavy chain, plus signal sequence
MKHLWFFLLLVAAPRWVLSQVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARGIFFAIWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 460 aa, Artificial Sequence
  Predicted protein sequence of the heavy chain of anti-NOTCH2/3 59RGV (germlined variant of 59R1), plus signal sequence
MKHLWFFLLLVAAPRWVLSEVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARGIFFAIWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 235 aa, Artificial Sequence
  Predicted protein sequence of anti-NOTCH2/3 59R1 light chain, plus signal sequence
MVLQTQVFISLLLWISGAYGDIVLTQSPATLSLSPGERATLSCRASQSVRSNYLAWYQQKPGQAPRLLIYGASSRATGVPARFSGSGSGTDFTLTISSLEPEDFAVYYCQQYSNFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 236 aa, Artificial Sequence
  Predicted protein sequence of the light chain of anti-NOTCH2/3 59RGV antibody (germlined variant of 59R1), plus signal sequence
MVLQTQVFISLLLWISGAYGEIVLTQSPATLSLSPGERATLSCRRASQSVRSNYLAWYQQKPGQAPRLLIYGASSRATGIPARFSGSGSGTDFTLTISSLEPEDFAVYYCQQYSNFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 18 bp, Artificial Sequence
  Probe
aggcagagtggcgacctc

SEQ ID NO: 36 - DNA, 23 bp, Artificial Sequence
  probe
cgtccacgttcacttcacaattc

SEQ ID NO: 37 - DNA, 25 bp, Artificial Sequence
  probe
aacccaggaagacaggcacagtcgt

SEQ ID NO: 38 - DNA, 20 bp, Artificial Sequence
  probe
ctgggtttgagggtcagaat

SEQ ID NO: 39 - DNA, 20 bp, Artificial Sequence
  probe
gggcactggcagttataggt

SEQ ID NO: 40 - DNA, 20 bp, Artificial Sequence
  probe
tgacgccatccacgcatgtc

SEQ ID NO: 41 - DNA, 22 bp, Artificial Sequence
  probe
tgcaggatagcaaggaggagac

SEQ ID NO: 42 - DNA, 20 bp, Artificial Sequence
  probe
gcagcttggcagcctcatag

SEQ ID NO: 43 - DNA, 23 bp, Artificial Sequence
  probe
ctcgcgggcggccaggaataggg

(19) United States
(12) Patent Application Publication
(10) Pub. No.: US 2016/0030561 A1
(43) Pub. Date: Feb. 4, 2016

(54) Methods of Treating Pancreatic Cancer

(71) Applicant: ONCOMED PHARMACEUTICALS, INC., Redwood City, CA (US)

(72) Inventors: Timothy Charles HOEY, Hillsborough, CA (US); Chun ZHANG, Palo Alto, CA (US); Ann M. KAPOUN, Mountain View, CA (US)

(73) Assignee: OncoMed Pharmaceuticals, Inc., Redwood City, CA (US)

(21) Appl. No.: 14/776,259

(22) PCT Filed: Mar. 13, 2014
(86) PCT No.: PCT/US14/26094

§ 371 Date: Sep. 14, 2015

Related U.S. Application Data
(60) Provisional application No. 61/794,788, filed on Mar. 15, 2013.

(51) Int. Cl. A61K 39/395; G01N 33/574; C12Q 1/68; C07K 16/28; A61K 45/06

(52) U.S. Cl. CPC A61K 39/39558; C07K 16/28; A61K 45/06; C12Q 1/6886; G01N 33/57438; C07K 2317/76; A61K 2039/505

(57) ABSTRACT

Novel methods of treating pancreatic cancer are provided. In one embodiment, the method comprises determining NOTCH mRNA expression levels in pancreatic cancer cells. In another embodiment, the method further comprises administering to a subject in need thereof a therapeutically effective dose of a NOTCH antagonist.

### CROSS REFERENCE TO RELATED APPLICATIONS

This application claims the priority benefit of U.S. Provisional Application No. 61/794,788, filed Mar. 15, 2013, which is hereby incorporated by reference herein in its entirety.

### FIELD OF THE INVENTION

The field of this invention generally relates to methods of treating pancreatic cancer. In one embodiment, the method comprises determining NOTCH gene expression levels in pancreatic cancer cells. In another embodiment, the method further comprises administering to a subject in need thereof a therapeutically effective dose of a NOTCH antagonist

### BACKGROUND OF THE INVENTION

The NOTCH signaling pathway is one of several critical regulators of embryonic pattern formation, post-embryonic tissue maintenance, and stem cell biology. Unregulated NOTCH signaling is associated with numerous human cancers where it can alter the developmental fate of tumor cells to maintain them in an undifferentiated and proliferative state (Brennan and Brown, 2003, Breast Cancer Res. 5:69). Thus, carcinogenesis can proceed by usurping homeostatic mechanisms controlling normal development and tissue repair by stem cell populations (Beachy et al., 2004, Nature 432:324).

The NOTCH receptor is a single-pass transmembrane receptor containing numerous tandem epidermal growth factor (EGF)-like repeats and three cysteine-rich NOTCH/LIN-12 repeats within a large extracellular domain (Wharton et al., 1985, Cell 43:567; Kidd et al., 1986, Mol. Cell Biol. 6:3094; reviewed in Artavanis et al., 1999, Science 284:770). Four mammalian NOTCH proteins have been identified (NOTCH1, NOTCH2, NOTCH3, and NOTCH4), and mutations in these receptors invariably result in developmental abnormalities and human pathologies including several cancers as described in detail below (Gridley, 1997, Mol. Cell Neurosci. 9:103; Joutel & Tournier-Lasserve, 1998, Semin. Cell Dev. Biol. 9:619-25).

Aberrant NOTCH signaling has been implicated in a number of human malignancies, for example, T-cell acute lymphoblastic leukemia, breast cancer, cervical cancer, renal cell carcinoma, head and neck squamous cell carcinoma. Aberrant NOTCH signaling has also been implicated in the development of pancreatic cancer. See, e.g., Mazur et al., Proc. Natl. Acad. Sci. USA. 107(30):13438-43 (2010), Wang et al., Cancer Res. 69(6):2400-7 (2009), Doucas et al., J. Surg. Oncol. 97(1):63-8 (2008), Yao and Qian, Med. Oncol. 27(3):1017-22 (2010); and Gungor et al., Cancer Res. 71(14):5009-19 (2011).

Pancreatic cancer is the fourth leading cause of cancer deaths with a median survival of 6 months and a dismal 5-year survival rate of 3-5% and this figure has remained relatively unchanged over the past 25 years (Iovanna et al., Front. Oncol. 2012; 2: 6). Even for patients diagnosed with local disease, the 5-year survival rate is only 15%. The lethal nature of pancreatic cancer stems from its propensity to rapidly disseminate to the lymphatic system and distant organs. The presence of occult or clinical metastases at the time of diagnosis together with the lack of effective chemotherapies contributes to the high mortality in patients with pancreatic cancer.

Pancreatic cancer is one of the most intrinsically drug-resistant tumors and resistance to chemotherapeutic agents is a major cause of treatment failure in pancreatic cancer. Gemcitabine is the standard chemotherapeutic drug for patients with advanced pancreatic cancer (Burris et al., Eur. J. Cancer 1997, 33:S18-22). Recently, a polychemotherapy regimen combining 5-FU, irinotecan, and oxaliplatin (FOLFIRINOX) was shown to nearly double overall survival compared to gemcitabine, at the expense of a manageable but increased toxicity, limiting its use to good performance status patients. In addition, overall survival was less than 12 months (Conroy et al., N. Engl. J. Med. 2011, 364:1817-25). Therefore, there is a need for designing new and targeted therapeutic strategies that can overcome the drug-resistance and improve the clinical outcome for patients diagnosed with pancreatic cancer.

### SUMMARY OF THE INVENTION

In one aspect, the invention provides methods for selecting a pancreatic cancer patient for treatment with a NOTCH inhibitor comprising: (a) determining the level of expression of one or more biomarkers in tumor cells from said patient, wherein the one or more biomarkers comprise NOTCH3, and (b) selecting the patient based on the expression level of the one or more biomarkers.

In another aspect, the invention provides methods for determining whether a patient diagnosed with pancreatic cancer is likely to respond to a NOTCH inhibitor-based therapy comprising determining the level of expression of one or more biomarkers in tumor cells from said patient, wherein the one or more biomarkers comprise NOTCH3, and the level of expression of the one or more biomarkers indicates that the patient is likely to respond to therapy.

In another aspect, the invention provides methods for determining whether a patient diagnosed with pancreatic cancer should be administered a NOTCH inhibitor, comprising determining the level of expression of one or more biomarkers in tumor cells from said patient, wherein the one or more biomarkers comprise NOTCH3, and the level of expression of the one or more biomarkers is predictive of said patient having a favorable response to treatment with a NOTCH inhibitor.

In another aspect, the invention provides methods to determine whether a patient diagnosed with pancreatic cancer should continue treatment with a NOTCH inhibitor, comprising determining the level of expression of one or more biomarkers in tumor cells from said patient, wherein the one or more biomarkers comprise NOTCH3, and the level of expression of the one or more biomarkers indicates that the patient is likely to respond to therapy.

In another aspect, the invention provides methods to determine whether a patient diagnosed with pancreatic cancer should continue treatment with a NOTCH inhibitor, comprising determining the level of expression of one or more biomarkers in tumor cells from said patient, wherein the one or more biomarkers comprise NOTCH3, and the level of expression of the one or more biomarkers is predictive of said patient having a favorable response to treatment with said NOTCH inhibitor.

In another aspect, the invention provides methods for determining the therapeutic efficacy of a NOTCH inhibitor for treating pancreatic cancer in a patient comprising determining the level of expression of one or more biomarkers in tumor cells from said patient, wherein the one or more biomarkers comprise NOTCH3, and the level of expression of the one or more biomarkers is indicative of the therapeutic efficacy of said NOTCH inhibitor.

In another aspect, the invention provides methods of treating pancreatic cancer in a patient comprising: (a) determining the level of expression of one or more biomarkers in tumor cells from said patient, wherein the one or more biomarkers comprise NOTCH3 and (b) administering to said patient a therapeutically effective amount of a NOTCH inhibitor.

In another aspect, the invention provides methods for stratifying a pancreatic cancer patient population for treatment with a NOTCH inhibitor comprising: (a) determining the level of expression of one or more biomarkers in tumor cells from said patients, wherein the one or more biomarkers comprise NOTCH3, and (b) stratifying the patient population based on the level of expression of the one or more biomarkers in the tumor cells.

In certain embodiments, the level of NOTCH3 expression is determined to be above a reference level for NOTCH3 expression. In certain embodiments, each of the biomarkers is determined to be expressed at a level above a reference level for the biomarker.

In certain embodiments, the expression level of the one or more biomarkers is determined by determining the level of the biomarker' mRNA or the biomarker protein. In certain embodiments, the level of NOTCH3 expression is determined by determining the level of NOTCH3 mRNA in the tumor cells. In certain embodiments, the NOTCH3 mRNA level is determined by quantitative polymerase chain reaction. In certain embodiments, the NOTCH3 mRNA level is determined using: (a) a forward primer having a nucleotide sequence selected from the group consisting of SEQ ID NO:35, SEQ ID NO:38, and SEQ ID NO:41; (b) a reverse primer having a nucleotide sequence selected from the group consisting of SEQ ID NO:36, SEQ ID NO:39, and SEQ ID NO:42; and/or (c) a probe comprising an oligonucleotide having a nucleotide sequence selected from the group consisting of SEQ ID NO:37, SEQ ID NO:40, and SEQ ID NO:43. In certain embodiments, the NOTCH3 mRNA level is determined using: (a) a forward primer having the sequence of SEQ ID NO:35, a reverse primer having the sequence of SEQ ID NO:36, and a probe comprising an oligonucleotide having the sequence of SEQ ID NO:37; (b) a forward primer having the sequence of SEQ ID NO:38, a reverse primer having the sequence of SEQ ID NO:39, and a probe comprising an oligonucleotide having the sequence of SEQ ID NO:40; or (c) a forward primer having the sequence of SEQ ID NO:41, a reverse primer having the sequence of SEQ ID NO:42, and a probe comprising an oligonucleotide having the sequence of SEQ ID NO:43. In certain embodiments, the NOTCH3 mRNA level is determined by array hybridization. In certain embodiments, the level of NOTCH3 expression is determined by determining the level of NOTCH3 protein expressed by the tumor cells.

In certain embodiments, the one or more biomarkers consist of NOTCH3. In certain embodiments, the one or more biomarkers further comprise MAML2 and the level of MAML2 expression is determined to be above a reference level for MAML2 expression. In certain embodiments, the one or more biomarkers consist of NOTCH3 and MAML2. In certain embodiments, the level of MAML2 expression is determined by determining the level of MAML2 mRNA in the tumor cells. In certain embodiments, the level of MAML2 expression is determined by determining the level of MAML2 protein expressed by the tumor cells.

In another aspect, the invention provides methods of treating pancreatic cancer in a patient comprising administering to said patient a therapeutically effective amount of a NOTCH inhibitor, wherein at least some of the pancreatic tumor cells from said patient express each of one or more biomarkers at a level above a reference level for that biomarker and/or have been previously determined to express each of one or more biomarkers at a level above a reference level for that biomarker, wherein the one or more biomarkers comprise NOTCH3. In certain embodiments, the level of NOTCH3 expression is determined as the level of NOTCH3 mRNA. In certain embodiments, the level of NOTCH3 expression is determined as the level of NOTCH3 protein. In certain embodiments, the one or more biomarkers consist of NOTCH3. In certain embodiments, the one or more biomarkers further comprise MAML2 and the level of MAML2 expression is above a reference level for MAML2 expression. In certain embodiments, the one or more biomarkers consist of NOTCH3 and MAML2.

In certain embodiments of the methods described herein, the reference level of a biomarker is a predetermined value. In certain embodiments, the reference level of a biomarker is the level of expression of that biomarker in a control sample. In certain embodiments, the reference level for NOTCH3 expression is the 25th percentile, the 30th percentile, the 40th percentile, the 50th percentile, the 60th percentile, the 70th percentile, the 75th percentile, or the 80th percentile for NOTCH3 expression in pancreatic cancers or a subset of pancreatic cancers. In certain embodiments, the reference level for NOTCH3 expression is the 75th percentile for NOTCH3 expression in pancreatic cancers. In certain embodiments, the reference level for NOTCH3 expression is the 50th percentile for NOTCH3 expression in pancreatic cancers. In certain embodiments, the reference level for NOTCH3 expression is the 25th percentile for NOTCH3 expression in pancreatic cancers. In certain embodiments, the reference level for NOTCH3 expression is the 75th percentile for NOTCH3 expression in pancreatic adenocarcinomas, metastatic pancreatic tumors, liver and/or lymph node metastatic pancreatic tumors, or chemotherapy-resistant pancreatic cancers. In certain embodiments, the reference level for NOTCH3 expression is the 50th percentile for NOTCH3 expression in pancreatic adenocarcinomas, metastatic pancreatic tumors, liver and/or lymph node metastatic pancreatic tumors or chemotherapy-resistant pancreatic cancers. In certain embodiments, the reference level for NOTCH3 expression is the 25th percentile for NOTCH3 expression in pancreatic adenocarcinomas, metastatic pancreatic tumors, liver and/or lymph node metastatic pancreatic tumors or chemotherapy-resistant pancreatic cancers.

In certain embodiments, a method described herein further comprises obtaining a body sample from said patient. In certain embodiments, the level of expression of NOTCH3 is the level in a body sample from the patient. In certain embodiments, the sample is whole blood, plasma, serum, or tissue. In certain embodiments, the sample is a pancreatic tumor sample. In certain embodiments, the sample is from a pancreatic tumor that has metastasized to the liver. In certain embodiments, the sample is formalin-fixed paraffin embedded (FFPE) tissue.

In certain embodiments of the methods described herein, the patient is a human or said patient population is a human population.

In certain embodiments of the methods described herein, the pancreatic cancer is adenocarcinoma. In certain embodiments, the pancreatic cancer is chemotherapy-resistant.

In certain embodiments, a method described herein comprises administering the NOTCH inhibitor to said patient. In certain embodiments, the NOTCH inhibitor is a gamma-secretase inhibitor. In certain embodiments, the NOTCH inhibitor is, an anti-NOTCH antibody.

In certain embodiments, the anti-NOTCH antibody specifically binds to human NOTCH2 or human NOTCH3. In certain embodiments, the anti-NOTCH antibody specifically binds to human NOTCH2 and NOTCH3. In certain embodiments, the anti-NOTCH antibody specifically binds to EGF repeat 10 of human NOTCH2. In certain embodiments, the anti-NOTCH antibody specifically binds to EGF repeat 9 of human NOTCH3. In certain embodiments, the anti-NOTCH antibody comprises an antigen-binding site that binds both the EGF repeat 9 of human NOTCH3 and the EGF repeat 10 of NOTCH2.

In certain embodiments, the NOTCH inhibitor is an antagonist of human NOTCH2 and/or NOTCH3. In certain embodiments, the NOTCH inhibitor inhibits binding of a ligand to human NOTCH2 and/or NOTCH3. In certain embodiments, the NOTCH inhibitor inhibits signaling of human NOTCH2 and/or NOTCH3.

In certain embodiments, the anti-NOTCH antibody is encoded by the polynucleotide deposited with ATCC as PTA-9547.

In certain embodiments, the anti-NOTCH antibody specifically binds human NOTCH2 and/or NOTCH3, wherein the antibody comprises (a) a heavy chain CDR1 comprising SSSGMS (SEQ ID NO:3), a heavy chain CDR2 comprising VIASSGSNTYYADSVKG (SEQ ID NO:4), and a heavy chain CDR3 comprising SIFYTT (SEQ ID NO:9); and (b) a light chain CDR1 comprising RASQSVRSNYLA (SEQ ID NO:6), a light chain CDR2 comprising GASSRAT (SEQ ID NO:7), and a light chain CDR3 comprising QQYSNFPI (SEQ ID NO:8). In certain embodiments, the anti-NOTCH antibody specifically binds human NOTCH2 and/or NOTCH3, wherein the antibody comprises (a) a heavy chain CDR1 comprising SSSGMS (SEQ ID NO:3), a heavy chain CDR2 comprising VIASSGSNTYYADSVKG (SEQ ID NO:4), and a heavy chain CDR3 comprising GIFFAI (SEQ ID NO:5); and (b) a light chain CDR1 comprising RASQSVRSNYLA (SEQ ID NO:6), a light chain CDR2 comprising GASSRAT (SEQ ID NO:7), and a light chain CDR3 comprising QQYSNFPI (SEQ ID NO:8).

In certain embodiments, the anti-NOTCH antibody specifically binds human NOTCH2 and/or NOTCH3, wherein the antibody comprises: (a) a heavy chain variable region having at least about 90% sequence identity to SEQ ID NO:17, SEQ ID NO:18, or SEQ ID NO:26; and (b) a light chain variable region having at least about 90% sequence identity to SEQ ID NO:29 or SEQ ID NO:27. In certain embodiments, the anti-NOTCH antibody comprises: (a) a heavy chain variable region having at least about 95% sequence identity to SEQ ID NO:17; and (b) a light chain variable region having at least about 95% sequence identity to SEQ ID NO:29. In certain embodiments, the anti-NOTCH antibody comprises: (a) a heavy chain variable region having at least about 95% sequence identity to SEQ ID NO:18; and (b) a light chain variable region having at least about 95% sequence identity to SEQ ID NO:29. In certain embodiments, the anti-NOTCH antibody comprises: (a) a heavy chain variable region comprising SEQ ID NO:18; and (b) a light chain variable region comprising SEQ ID NO:29. In certain embodiments, the anti-NOTCH antibody comprises: (a) a heavy chain variable region comprising SEQ ID NO:17; and (b) a light chain variable region comprising SEQ ID NO:29.

In certain embodiments, the anti-NOTCH antibody competes for specific binding to human NOTCH2 and/or NOTCH3 with an antibody selected from the group consisting of (a) an antibody comprising a heavy chain variable region comprising SEQ ID NO:17 or SEQ ID NO:18, and a light chain variable region comprising SEQ ID NO:29; (b) an antibody comprising a heavy chain CDR1 comprising SSSGMS (SEQ ID NO:3), a heavy chain CDR2 comprising VIASSGSNTYYADSVKG (SEQ ID NO:4), and a heavy chain CDR3 comprising SIFYTT (SEQ ID NO:9), and a light chain CDR1 comprising RASQSVRSNYLA (SEQ ID NO:6), a light chain CDR2 comprising GASSRAT (SEQ ID NO:7), and a light chain CDR3 comprising QQYSNFPI (SEQ ID NO:8); and (c) an antibody encoded by the polynucleotide deposited with ATCC as PTA-9547.

In certain embodiments, the anti-NOTCH antibody is a monoclonal antibody. In certain embodiments, the anti-NOTCH antibody is a chimeric antibody, a humanized antibody, a human antibody, or an antibody fragment.

In certain embodiments, a method described herein further comprises administering a second therapeutic agent. In certain embodiments, the second therapeutic agent is a chemotherapeutic agent. In certain embodiments, the second therapeutic agent is a nucleoside analogue or a mitotic inhibitor. In certain embodiments, the second therapeutic agent is gemcitabine, paclitaxel, albumin-bound paclitaxel, or combinations thereof.

In another aspect, the invention provides a diagnostic composition comprising an isolated polynucleotide comprising a sequence selected from the group consisting of SEQ ID NO:35-43. In certain embodiments, the diagnostic composition comprises: (a) a polynucleotide having the sequence of. SEQ ID NO:35, a polynucleotide having the sequence of SEQ ID NO:36, and a polynucleotide having the sequence of SEQ ID NO:37; (b) a polynucleotide having the sequence of SEQ ID NO:38, a polynucleotide having the sequence of SEQ ID NO:39, and a polynucleotide having the sequence of SEQ ID NO:40; or (c) a polynucleotide having the sequence of SEQ ID NO:41, a polynucleotide having the sequence of SEQ ID NO:42, and a polynucleotide having the sequence of SEQ ID NO:43.

In another aspect, the invention provides methods of detecting NOTCH3 mRNA in a sample, comprising contacting the sample with a polynucleotide comprising a sequence selected from the group consisting of SEQ ID NO:35-43. In certain embodiments, the method comprises contacting the sample with (a) a forward primer having the sequence of SEQ ID NO:35, a reverse primer having the sequence of SEQ ID NO:36, and a probe comprising an oligonucleotide having the sequence of SEQ ID NO:37; (b) a forward primer having the sequence of SEQ ID NO:38, a reverse primer having the sequence of SEQ ID NO:39, and a probe comprising an oligonucleotide having the sequence of SEQ ID NO:40; or (c) a forward primer having the sequence of SEQ ID NO:41, a reverse primer having the sequence of SEQ ID NO:42, and a probe comprising an oligonucleotide having the sequence of SEQ ID NO:43.

In another aspect, the invention provides kits for detecting NOTCH3 mRNA in a sample, comprising a polynucleotide comprising a sequence selected from the group consisting of SEQ ID NO:35-43. In certain embodiments, the kit comprises: (a) a polynucleotide having the sequence of SEQ ID NO:35, a polynucleotide having the sequence of SEQ ID NO:36, and a polynucleotide having the sequence of SEQ ID NO:37; (b) a polynucleotide having the sequence of SEQ ID NO:38, a polynucleotide having the sequence of SEQ ID NO:39, and a polynucleotide having the sequence of SEQ ID NO:40; or (c) a polynucleotide having the sequence of SEQ ID NO:41, a polynucleotide having the sequence of SEQ ID NO:42, and a polynucleotide having the sequence of SEQ ID NO:43.

In another aspect, the invention provides primers having a sequence selected from the group consisting of SEQ ID NO:35, SEQ ID NO:36, SEQ ID NO:38, SEQ ID NO:39, SEQ ID NO:41, and SEQ ID NO:42.

In another aspect, the invention provides probes comprising an oligonucleotide having a sequence selected from the group consisting of SEQ ID NO:37, SEQ ID NO:40, and SEQ ID NO:43.

### BRIEF DESCRIPTIONS OF THE DRAWINGS

FIG. 1. Activity of OMP-59R5 as a single agent, or in combination with a chemotherapeutic agent in (FIG. 1A) PN8 pancreatic tumor cells, (FIG. 1B) PN17 pancreatic tumor cells, (FIG. 1C) PN11 pancreatic tumor cells, (FIG. 1D) UM-PE13 breast tumor cells, (FIG. 1E) UM-T1 breast tumor cells, (FIG. 1F) OMP-Lu40 lung tumor cells, and (FIG. 1G) OMP-Lu53 lung tumor cells.

FIG. 2. Correlation of NOTCH3 gene expression and OMP-59R5 tumor inhibition. (FIG. 2A) Extent of pancreatic tumor inhibition by the OMP-59R5 antibody, in combination with gemcitabine, significantly correlates with the levels of NOTCH3 gene expression in the pancreatic tumor cells. (FIG. 2B) Distribution of NOTCH3 gene expression in pancreatic tumors that are responsive (R) and non-responsive (NR) to OMP-59R5 antibody treatment in combination with gemcitabine. NOTCH3 gene expression distribution is shown as a boxplot depicting the sample minimum, lower quartile, median, upper quartile and sample maximum.

FIG. 3. NOTCH3 gene expression in pancreatic tumors that are responsive and non-responsive to OMP-59R5 antibody treatment, in combination with gemcitabine, as determined by RNAseq. NOTCH3 gene expression was measured as RPKM (Reads Per Kilobase of transcript per Million mapped reads).

FIG. 4. Predicted probability of response to OMP-59R5 antibody treatment, in combination with gemcitabine, in pancreatic tumors based on NOTCH3 gene expression as a predictive indicator.

FIG. 5. Predicted probability of response to OMP-59R5 antibody treatment, in combination with gemcitabine, in pancreatic tumors based on NOTCH3 and MAML2 gene expression as a predictive indicator.

FIG. 6. NOTCH3 expression in pancreatic tumors. (FIG. 6A) NOTCH3 gene and protein expression in pancreatic tumors. (FIG. 6B) Distribution of NOTCH3 protein expression in pancreatic tumors that are responsive (R) and non-responsive (NR) to OMP-59R5 antibody treatment in combination with gemcitabine. NOTCH3 protein expression distribution is shown as a boxplot depicting the sample minimum, lower quartile, median, upper quartile and sample maximum.

FIG. 7. NOTCH3 gene expression in pancreatic cancer metastatic tissues. NOTCH3 gene expression was measured by RT-PCR. NOTCH3 gene expression distribution is shown as a boxplot depicting the sample minimum, lower quartile, median, upper quartile and sample maximum observed within samples of a particular tumor type. Vertical dashed lines represent 10^{th}, 25^{th}, 50^{th}, 75^{th}, and 90^{th} percentile NOTCH3 expression values observed across all metastatic pancreatic tumor samples.

FIG. 8. NOTCH3 gene expression in liver and lymph node pancreatic cancer metastatic tissues, and xenografted tumors. NOTCH3 gene expression was measured by RT-PCR. NOTCH3 gene expression distribution is shown as a boxplot depicting the sample minimum, lower quartile, median, upper quartile and sample maximum observed within samples of a particular tumor type. Vertical dashed lines represent 10^{th}, 25^{th}, 50^{th}, 75^{th}, and 90^{th} percentile NOTCH3 expression values observed in the lymph node and liver metastatic pancreatic tumor samples.

FIG. 9. OMP-59R5 is active in combination with gemcitabine and ABRAXANE™ (protein bound paclitaxel) in pancreatic tumors.

### DETAILED DESCRIPTION OF THE INVENTION

The present invention is broadly directed to methods of treating pancreatic cancer using a NOTCH inhibitor. The invention provides methods for stratifying a pancreatic cancer patient population for treatment with a NOTCH inhibitor, methods for selecting a pancreatic patient for treatment with a NOTCH inhibitor, methods for determining whether a patient diagnosed with pancreatic cancer is likely to respond to a NOTCH inhibitor-based therapy, methods for determining whether a patient diagnosed with pancreatic cancer should be administered a NOTCH inhibitor, methods to determine whether a patient diagnosed with pancreatic cancer should continue treatment with a NOTCH inhibitor, and methods for determining the therapeutic efficacy of a NOTCH inhibitor for treating pancreatic cancer in a patient. In some embodiments, the methods comprise determining the level of NOTCH3 gene expression in tumor cells from a patient. In some embodiments, the methods provided herein further comprise determining the level of MAML2 gene expression in tumor cells from a patient. In some embodiments, the methods provided herein comprise administering a NOTCH inhibitor. In some embodiments, the NOTCH inhibitor is an antibody that specifically binds to one or binds to more than one human NOTCH receptor. In some embodiments, the antibody is administered in combination with a chemotherapeutic agent. In some embodiments, the chemotherapeutic agent is a nucleoside analogue or a mitotic inhibitor.

### 1. Definitions

To facilitate an understanding of the present invention, a number of terms and phrases are defined below.

“NOTCH” is a membrane-bound transcription factor that regulates many cellular processes, especially in development. In response to ligand binding, its intracellular domain (ICD) is released by two proteases. The released intracellular domain enters the nucleus and interacts with a DNA-bound protein to activate transcription. The extracellular domain of NOTCH and related proteins contains up to 36 EGF-like domains, followed by three notch (DSL) domains. The intracellular domain (ICD) contains six ankyrin repeats and a carboxyl-terminal extension that includes a PEST domain. The NOTCH1 and NOTCH2 ICDs additionally comprise a transactivation domain (TAD). “NOTCH” encompasses all members of the NOTCH receptor family. A description of the NOTCH signaling pathway and conditions affected by it can be found, for example, in WO 98/20142 and WO 00/36089.

There are four members of the NOTCH family in mammals: NOTCH1 (TAN1), NOTCH2, NOTCH3 and NOTCH4/Int-4. Exemplary sequences for the human NOTCH proteins include, but are not limited to: human NOTCH1 is encoded by the mRNA sequence set forth as Genbank Acc. No. NM_{—}017617.3, and has the amino acid sequence set forth as Genbank Acc. No. NP_{—}060087; human NOTCH2 is encoded by the mRNA sequence set forth as Genbank Acc. No. NM_{—}024408, and has the amino acid sequence set forth as Genbank Acc. No. NP_{—}077719; human NOTCH3 is encoded by the mRNA sequence of Genbank Acc. No. NM_{—}000435.2, and has the amino acid sequence of Genbank Acc. No. NP_{—}000426; and human NOTCH4 is encoded by the mRNA sequence of Genbank Acc. No. NM_{—}004557, and has the amino acid sequence of Genbank Acc. No. NP_{—}004548.

A “NOTCH inhibitor,” “NOTCH antagonist,” “anti-NOTCH therapeutic agent,” or “anti-NOTCH agent” as used herein includes any compound that partially or fully blocks, inhibits, or neutralizes a biological activity of the NOTCH pathway. Exemplary NOTCH inhibiting compounds include, but are not limited to gamma-secretase inhibitors such as, N-[N-(3,5-difluorophenacetyl)-L-alanyl]S-phenylglycine t-butyl ester) (RAPT), compound E, D-helical peptide 294, isocoumarins, BOC-Lys(Cbz)Ile-Leu-epoxide, and (Z-LL)2-ketone (see, Kornilova et al., J. Biol. Chem. 2003, 278:16479-16473); and those compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, WO 2004/089911, WO 02/081435, WO 02/081433, WO 03/018543, WO 2004/031137, WO 2004/031139, WO 2004/031138, WO 2004/101538, WO 2004/101539 and WO 02/47671 and U.S. Patent Application No. 2003/0114496. Specific gamma-secretase inhibitor compounds are also described in U.S. Pat. Nos. 6,984,663 and 7,304,094. Specific antibody NOTCH inhibitors are described herein, as well as in WO 2010/005566, and WO 2010/005567, all of which are herein incorporated by reference. NOTCH inhibitors also include NOTCH ligand antagonists.

“NOTCH inhibitors,” “NOTCH antagonists,” “anti-NOTCH therapeutic agents,” or “anti-NOTCH agents” also encompass antibodies that bind the NOTCH receptor. The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination, portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda, Md.)); and (2) an approach based on crystallographic studies of antigen-antibody complexes (Al-lazikani et al., J. Molec. Biol. 1997, 273:927-948)). In addition, combinations of these two approaches are sometimes used in the art to determine CDRs.

The term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody” refers to forms of non-human (e.g. murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g. mouse, rat, rabbit, hamster) that have the desired specificity, affinity, and capability (Jones et al., 1986, Nature 321:522-525; Riechmann et al., 1988, Nature 332:323-327; Verhoeyen et al., 1988, Science 239:1534-1536). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539.

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc.) with the desired specificity, affinity, and capability while the constant regions are homologous to the sequences in antibodies derived from another (usually human) to avoid eliciting an immune response in that species.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

That a polypeptide or other agent (e.g., antibody or soluble receptor) “specifically binds” to a protein means that the polypeptide or other agent reacts or associates more frequently, more rapidly, with greater duration, with greater affinity, or with some combination of the above to the protein than with alternative substances, including unrelated proteins. In certain embodiments, “specifically binds” means, for instance, that an agent (e.g., antibody or soluble receptor) binds to a protein with a K_{D} of about 0.1 mM or less, but more usually less than about 1 μM. In certain embodiments, “specifically binds” means that an agent (e.g., antibody or soluble receptor) binds to a protein at times with a K_{D} of at least about 0.1 μM or less, at least about 0.01 μM or less, and at other times at least about 1 nM or less. Because of the sequence identity between homologous proteins in different species, specific binding can include an agent (e.g., antibody or soluble receptor) that recognizes a particular protein such as a NOTCH receptor in more than one species. Likewise, because of homology between different paralogues (e.g., the different human NOTCH proteins) in certain regions of their sequences, specific binding can include a polypeptide or an agent (e.g., antibody or soluble receptor) that recognizes more than one paralogue (e.g., more than one human NOTCH protein). It is understood that an agent (e.g., antibody or soluble receptor) that specifically binds to a first target may or may not specifically bind to a second target. As such, “specific binding” does not necessarily require (although it can include) exclusive binding, i.e. binding to a single target. Thus, an agent (e.g., antibody or soluble receptor) may, in certain embodiments, specifically bind to more than one target (e.g., multiple different human NOTCH proteins, such as NOTCH1, NOTCH2, NOTCH3 and/or NOTCH4). In certain embodiments, the multiple targets of an antibody may be bound by the same antigen-binding site on the antibody. For example, an antibody may, in certain instances, comprise two identical antigen-binding sites, each of which specifically binds two or more human frizzled receptors (e.g., human NOTCH1, NOTCH2, NOTCH3 and/or NOTCH4). In certain alternative embodiments, an antibody may be bispecific and comprise at least two antigen-binding sites with differing specificities. By way of non-limiting example, a bispecific antibody may comprise one antigen-binding site that recognizes an epitope on one NOTCH receptor, such as human NOTCH2, and further comprises a second, different antigen-binding site that recognizes a different epitope on a second NOTCH receptor, such as human NOTCH3. Generally, but not necessarily, reference to binding means specific binding.

The terms “cancer” and “cancerous” refer to or describe the physiological condition in mammals in which a population of cells are characterized by unregulated cell growth. The term cancer is understood to encompass NOTCH-dependent cancers. Examples of cancer include, but are not limited to, carcinoma, lymphoma, blastoma, sarcoma, and leukemia.

“Tumor” and “neoplasm” refer to any mass of tissue that result from excessive cell growth or proliferation, either benign (noncancerous) or malignant (cancerous) including pre-cancerous lesions.

“Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer stem cell,” “tumor stem cell,” or “solid tumor stem cell” are used interchangeably herein and refer to a population of cells from a solid tumor that (1) have extensive proliferative capacity; 2) are capable of asymmetric cell division to generate one or more kinds of differentiated progeny with reduced proliferative or developmental potential; and (3) are capable of symmetric cell divisions for self-renewal or self-maintenance. These properties of “cancer stem cells,” “tumor stem cells,” or “solid tumor stem cells” confer on those cancer stem cells the ability to form palpable tumors upon serial transplantation into an immunocompromised mouse compared to the majority of tumor cells that fail to form tumors. Cancer stem cells undergo self-renewal versus differentiation in a chaotic manner to form tumors with abnormal cell types that can change over time as mutations occur.

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

The term “tumorigenic” refers to the functional features of a solid tumor stem cell including the properties of self-renewal (giving rise to additional tumorigenic cancer stem cells) and proliferation to generate all other tumor cells (giving rise to differentiated and thus non-tumorigenic tumor cells) that allow solid tumor stem cells to form a tumor. These properties of self-renewal and proliferation to generate all other tumor cells confer on cancer stem cells the ability to form palpable tumors upon serial transplantation into an immunocompromised mouse compared to non-tumorigenic tumor cells, which are unable to form tumors upon serial transplantation. It has been observed that non-tumorigenic tumor cells may form a tumor upon primary transplantation into an immunocompromised mouse after obtaining the tumor cells from a solid tumor, but those non-tumorigenic tumor cells do not give rise to a tumor upon serial transplantation.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject. A “normal” subject or sample from a “normal” subject as used herein for quantitative and qualitative data refers to a subject who has or would be assessed by a physician as not having pancreatic cancer.

A “control sample” means a separate sample from a control cell. The control cell can be disease free, or can be a pancreatic cancer cell. The control cell can be from the same subject or from another subject. The control cell can be from the same tissue or from a different tissue. The control cell can be from an immortalized cell line.

The term “prognosis” is used herein to refer to the prediction of the likelihood of cancer attributable to death or progression, including recurrence, metastatic spread, and drug resistance, of a neoplastic disease, such as pancreatic cancer. As used herein, the term “predicting” or “prediction” refers to making a finding that a subject has a significantly enhanced or reduced probability of an outcome—favorable prognosis versus an unfavorable prognosis. It can also include the likelihood that a NOTCH inhibitor may be therapeutically effective versus one that is not found to be therapeutic. The term may also be used to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention can be used clinically to make treatment decisions by choosing the most appropriate treatment modalities for any particular patient. Towards this end, the predictive methods of the present invention are valuable tools in predicting if a patient is likely to respond favorably to a NOTCH-based treatment regimen, such as anti-NOTCH antibody treatment, chemotherapy with a given drug or drug combination, e.g. gamma-secretase inhibitor or another NOTCH inhibitor, or whether long-term survival of the patient, following a treatment protocol with a NOTCH inhibitor and/or termination of chemotherapy or other treatment modalities is likely.

The term “therapeutically effective amount” refers to an amount of an agent (e.g., antibody, soluble receptor, polypeptide, polynucleotide, small organic molecule, or other drug) effective to “treat” a disease or disorder in a subject or mammal. In the case of cancer, the therapeutically effective amount of the agent can reduce the number of cancer cells; reduce the tumor size; inhibit or stop cancer cell infiltration into peripheral organs including, for example, the spread of cancer into soft tissue and bone; inhibit and stop tumor metastasis; inhibit and stop tumor growth; relieve to some extent one or more of the symptoms associated with the cancer; reduce morbidity and mortality; improve quality of life; decrease tumorigenicity, tumorigenic frequency, or tumorigenic capacity of a tumor; reduce the number or frequency of cancer stem cells in a tumor; differentiate tumorigenic cells to a non-tumorigenic state; or a combination of such effects. To the extent the agent prevents growth and/or kills existing cancer cells, it can be referred to as cytostatic and/or cytotoxic.

As used herein the term “inhibit tumor growth” refers to any mechanism by which tumor cell growth can be inhibited. In certain embodiments, tumor cell growth is inhibited by slowing proliferation of tumor cells. In certain embodiments, tumor cell growth is inhibited by halting proliferation of tumor cells. In certain embodiments, tumor cell growth is inhibited by killing tumor cells. In certain embodiments, tumor cell growth is inhibited by inducing apoptosis of tumor cells. In certain embodiments, tumor cell growth is inhibited by inducing differentiation of tumor cells. In certain embodiments, tumor cell growth is inhibited by depriving tumor cells of nutrients. In certain embodiments, tumor cell growth is inhibited by preventing migration of tumor cells. In certain embodiments, tumor cell growth is inhibited by preventing invasion of tumor cells.

As used herein, the term “stratifying” refers to sorting subjects into different classes or strata based on the features of a particular disease state or condition. For example, stratifying a population of subjects with pancreatic cancer involves assigning the subjects based on the NOTCH3 gene expression levels in the tumor cells and/or on the basis of the severity of the disease (e.g., pre-malignant, malignant, metastatic etc.).

Terms such as “treating,” or “treatment,” or “to treat,” or “alleviating,” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for cancer according to the methods of the present invention if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; or some combination of effects.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

As used herein, the terms “biopsy” or “biopsy tissue” refer to a sample of tissue or fluid that is removed from a subject for the purpose of determining if the sample contains cancerous tissue. In some embodiments, biopsy tissue or fluid is obtained because a subject is suspected of having cancer. The biopsy tissue or fluid is then examined for the presence or absence of cancer.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; Band C; A (alone); B (alone); and C (alone).

### 2. NOTCH3 Evaluation Methods

As shown in detail below, the sensitivity of human pancreatic tumors to the anti-NOTCH2/3 antibody OMP-59R5 significantly correlated with increased NOTCH3 expression. Surprisingly, while both NOTCH3 mRNA and protein expression correlated with OMP-59R5 sensitivity in human pancreatic tumors, the correlation was increased between NOTCH3 mRNA expression and treatment sensitivity than between NOTCH3 protein expression and treatment sensitivity. These data strikingly contrast the expression data from human breast, tumor and colon tumors which showed that there was no significant correlation between either NOTCH2 or NOTCH3 expression and tumor sensitivity to OMP-59R5 treatment. Similarly, no correlation between OMP-59R5 sensitivity and NOTCH2 expression was seen in human pancreatic tumors.

The correlation between increased or elevated NOTCH3 expression (e.g., NOTCH3 over-expression) and sensitivity to OMP-59R5 treatment in pancreatic cancers (therapeutic efficacy) can be exploited to improve methods of treating pancreatic cancer by selecting pancreatic cancer patients for OMP-59R5 therapy whose tumor cells are characterized by elevated or increased NOTCH3 expression, NOTCH3 overexpression or NOTCH3 expression at or above a predetermined level. The terms “elevated NOTCH3 expression,” “increased NOTCH3 expression,” and “NOTCH3 overexpression” are, in some instances, used interchangeably herein. Therapeutic efficacy can also be improved by not selecting pancreatic cancer patients for OMP-59R5 therapy whose tumor cells are characterized by normal or reduced NOTCH3 expression, or NOTCH3 expression below a predetermined level. In certain embodiments, the predetermined NOTCH3 expression level can be the level of expression in a control sample, e.g., control cell. In certain embodiments, the predetermined NOTCH3 expression level can be the median level of NOTCH3 expression in pancreatic cancers, or the 95^{th}, 90^{th}, 80^{th}, 75^{th}, 70^{th}, 50^{th}, 40^{th}, 30^{th}, 25^{th} or 10^{th} percentile for NOTCH3 expression in pancreatic cancers.

In certain embodiments, a patient has a pancreatic tumor in which at least some of the tumor cells demonstrate elevated NOTCH3 expression levels. In one embodiment, elevated NOTCH3 expression level is a level at or above the median level of NOTCH3 expression in pancreatic cancers. In another embodiment, elevated NOTCH3 expression level is a level that is at or above the 95^{th}, 90^{th}, 80^{th}, 75^{th}, 70^{th}, 50^{th}, 40^{th}, 30^{th}, 25^{th} or 10^{th} percentile for NOTCH3 gene expression of pancreatic cancers. In certain embodiments, the median level of NOTCH3 expression of pancreatic cancers is the median level of NOTCH3 expression of pancreatic adenocarcinomas, metastatic pancreatic cancers, liver and/or lymph node metastatic pancreatic cancers, chemotherapy-resistant pancreatic cancers, or advanced, refractory or recurrent pancreatic cancers. In certain embodiments, the 95^{th}, 90^{th}, 80^{th}, 75^{th}, 70^{th}, 50^{th}, 40^{th}, 30^{th}, 25^{th} or 10^{th} percentile for NOTCH3 expression in pancreatic cancers is the 95^{th}, 90^{th}, 80^{th}, 75^{th}, 70^{th}, 50^{th}, 40^{th}, 30^{th}, 25^{th} or 10^{th} percentile for NOTCH3 expression in pancreatic adenocarcinomas, metastatic pancreatic cancers, liver and/or lymph node metastatic pancreatic cancers, chemotherapy-resistant pancreatic cancers, or advanced, refractory or recurrent pancreatic cancers. 10821 In certain embodiments, elevated NOTCH3 expression level is a level that is at or above a predetermined standard level, or reference level, or control level. The terms “predetermined standard,” “reference level,” and “control level” are, in some instances, used interchangeably herein. In one embodiment, a predetermined standard demonstrates NOTCH3 expression levels as measured in a control sample, e.g., a sample containing pancreatic cells that does not comprise pancreatic tumor or pancreatic cancer cells. In another embodiment, a predetermined standard demonstrates NOTCH3 expression levels as measured in a sample comprising pancreatic tumor cells, e.g., adenocarcinomas, metastatic tumor cells and liver or lymph node metastatic tumor cells. In a further embodiment, a predetermined standard demonstrates NOTCH3 expression levels as measured in a sample comprising pancreatic cancer cells that do not respond to treatment with a NOTCH inhibitor, e.g., OMP-59R5. In a further embodiment, a predetermined standard demonstrates NOTCH3 expression levels as measured in a sample comprising pancreatic cancer cells that respond to treatment with a NOTCH inhibitor, e.g., OMP-59R5. In another embodiment, a predetermined standard is NOTCH3 expression levels in an isolated cell line. The cell line can be derived from a pancreatic cancer sample. The cell line can also be recombinantly manipulated to express NOTCH3. In certain embodiments, a predetermined standard or reference level for NOTCH3 expression is the 95^{th}, 90^{th}, 80^{th}, 75^{th}, 70^{th}, 50^{th}, 40^{th}, 30^{th}, 25^{th} or 10^{th} percentile for NOTCH3 gene expression in pancreatic cancers, for example, in pancreatic adenocarcinomas, metastatic pancreatic tumors, liver and/or lymph node metastatic pancreatic tumors, chemotherapy-resistant pancreatic cancers, or advanced, refractory or recurrent pancreatic cancers.

In certain embodiments, a patient is selected for treatment and/or treated with a NOTCH inhibitor (e.g., OMP-59R5) when at least some of the patient's pancreatic tumor cells express NOTCH3 at an elevated level. In certain embodiments, at least some of the patient's pancreatic tumor cells express NOTCH3 at a level that is at or above a reference level. In certain embodiments, at least some of the patient's pancreatic tumor cells express NOTCH3 at a level that is at or above the median level of NOTCH3 expression in pancreatic cancers. In certain embodiments, at least some of the patient's pancreatic tumor cells express NOTCH3 at a level that is at or above the 95^{th}, 90^{th}, 80^{th}, 75^{th}, 70^{th}, 50^{th}, 40^{th}, 30^{th}, 25^{th} or 10^{th} percentile for NOTCH3 gene expression of pancreatic cancers. In certain embodiments, at least some of the patient's pancreatic tumor cells express NOTCH3 at a level that is at or above the 25^{th} percentile for NOTCH3 gene expression of pancreatic cancers. In certain embodiments, at least some of the patient's pancreatic tumor cells also express MAML2 at a level that is at or above a reference level, or at or above the median level of MAML2 expression in pancreatic cancers. In one embodiment, the patient is selected for treatment and/or treated with OMP-59R5. In another embodiment, the patient is selected for treatment and/or treated with an antibody comprising the six CDRs and/or the variable regions of OMP-59R5.

In certain embodiments, a patient is selected for treatment and/or treated with a NOTCH inhibitor (e.g., OMP-59R5) when at least some of the patient's pancreatic tumor cells comprise a level of NOTCH3 mRNA at or above (1) a reference level, (2) the median level of NOTCH3 mRNA in pancreatic cancers; and/or (3) the 95^{th}, 90^{th}, 80^{th}, 75^{th}, 70^{th}, 50^{th}, 40^{th}, 30^{th}, 25^{th} or 10^{th} percentile for NOTCH3 mRNA level in pancreatic cancers. In a particular embodiment, at least some of the patient's pancreatic tumor cells comprise a level of NOTCH3 mRNA at or above the 25^{th} percentile for NOTCH3 mRNA level in pancreatic cancers, e.g., in liver and/or lymph-node metastatic pancreatic cancers. In certain embodiments, at least some of the patient's pancreatic tumor cells also comprise MAML2 mRNA at or above a reference level, or at or above the median level of MAML2 mRNA in pancreatic cancers. In one embodiment, the patient is selected for treatment and/or treated with OMP-59R5. In another embodiment, the patient is selected for treatment and/or treated with an antibody comprising the six CDRs and/or the variable regions of OMP-59R5.

In certain embodiments, a patient is selected for treatment and/or treated with a NOTCH inhibitor (e.g., OMP-59R5) when at least some of the patient's pancreatic tumor cells comprise a level of NOTCH3 protein at or above (1) a reference level, (2) the median level of NOTCH3 protein in pancreatic cancers; and/or (3) the 95^{th}, 90^{th}, 80^{th}, 75^{th}, 70^{th}, 50^{th}, 40^{th}, 30^{th}, 25^{th} or 10^{th} percentile for NOTCH3 protein level in pancreatic cancers. In a particular embodiment, at least some of the patient's pancreatic tumor cells comprise a level of NOTCH3 protein at or above the 25^{th} percentile for NOTCH3 protein level in pancreatic cancers, e.g., in liver and/or lymph-node metastatic pancreatic cancers. In certain embodiments, at least some of the patient's pancreatic tumor cells also comprise MAML2 protein at or above a reference level, or at or above the median level of MAML2 protein in pancreatic cancers. In one embodiment, the patient is selected for treatment and/or treated with OMP-59R5. In another embodiment, the patient is selected for treatment and/or treated with an antibody comprising the six CDRs and/or the variable regions of OMP-59R5.

Methods for detecting the level of NOTCH3 or the expression of another gene/gene product of interest (e.g., MAML2) comprise any method capable of determining the level of NOTCH3 expression at either the nucleic acid or protein level. Such methods are well known in the art and include, but are not limited to Western blots, enzyme-linked immunosorbent assay (ELISA), immunoprecipitation, immunofluorescence, flow cytometry, immunohistochemistry (IHC), nucleic acid hybridization techniques, nucleic acid reverse transcription methods, nucleic acid amplification methods such as PCR or qRT-PCR, RNase protection, microarrays, serial analysis of gene expression (SAGE), high-throughput mass spectrometry (MS), whole transcriptome shotgun sequencing (WTSS), massively parallel signature sequencing (MPSS), in situ hybridization, and Northern blotting.

The median or percentile expression level of NOTCH3 in pancreatic cancers can be determined at any time relative to measuring NOTCH3 expression in a patient's pancreatic tumor cells. In certain embodiments, the NOTCH3 expression levels are measured contemporaneously. In another embodiment, the median or percentile expression level of NOTCH3 in pancreatic cancers is determined prior to measurement of the NOTCH3 expression level in a patient's sample.

In one embodiment, NOTCH3 expression is measured in a body sample. The phrase “body sample” as used herein, is intended any sample comprising a cell, a tissue, or a bodily fluid in which the level of NOTCH3 expression can be detected. Examples of such body samples include, but are not limited to, blood, lymph, urine, gynecological fluids, biopsies, amniotic fluid and smears. Body samples can be obtained from a patient by a variety of techniques. Methods for collecting various body samples are well known in the art. In certain embodiments, the body sample is a pancreatic tumor sample. In certain embodiments, the body sample can be a fixed sample, e.g. a formalin fixed, paraffin-embedded (FFPE) sample, or a frozen sample.

In particular embodiments, the level of NOTCH3 expression is detected at the mRNA level. Various methods for determining expression of mRNA include, but are not limited to, quantitative real time PCR (qRT-PCR), microarray analysis, serial analysis of gene expression (SAGE), etc. In certain embodiments, the mRNA level in pancreatic tumor cells is determined using quantitative real time PCR (qRT-PCR) or microarray analysis. Many expression detection methods use isolated RNA. Any RNA isolation technique that does not select against the isolation of mRNA can be utilized for the purification of RNA from body samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski (U.S. Pat. No. 4,843,155).

The term “probe” refers to any molecule that is capable of selectively binding to a specifically intended target biomolecule, for example, a nucleotide transcript of NOTCH3. Probes can be synthesized by one of skill in the art using known techniques, or derived from appropriate biological preparations. Probes can be specifically designed to be labeled with a detectable label. Examples of molecules that can be used as probes include, but are not limited to, RNA, DNA, proteins (including peptides), antibodies, and organic molecules.

NOTCH3 mRNA from pancreatic tumor cells can be detected in hybridization or amplification assays that include, but are not limited to, mRNA sequencing methods, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding NOTCH3. Hybridization of an mRNA with the probe indicates that the gene in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array (Santa Clara, Calif.). Known mRNA detection methods can be readily adapted for use in determining NOTCH3 mRNA in pancreatic tumor cells.

An alternative method for determining the level of NOTCH3 mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189 193), self sustained sequence replication (Guatelli, 1990, Proc. Natl. Acad. USA, 87:1874 1878), transcriptional amplification system (Kwoh, 1989, Proc. Natl. Acad. Sci. USA, 86:1173 1177), Q-Beta Replicase (Lizardi, 1988, Bio/Technology, 6:1197), rolling circle replication (Lizardi, U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of NOTCH3 mRNA is assessed by quantitative fluorogenic RT-PCR (i.e., the TaqMan® System). Such methods typically use pairs of oligonucleotide primers that flank introns within the NOTCH3 gene. Methods for designing oligonucleotide primers specific for a known sequence are known in the art.

In one embodiment, the present invention provides primer sets that are suitable for determining the level of NOTCH3 mRNA in a sample using quantitative RT-PCR. In one embodiment, the primer set comprises three isolated polynucleotides comprising the sequence of SEQ ID NO:35, 36, and 37. In one embodiment the primer set comprises three isolated polynucleotides comprising the sequence of SEQ ID NO:38, 39, and 40. In one embodiment, the primer set comprises three isolated polynucleotides comprising the sequence of SEQ ID NO:41, 42, and 43. In a further aspect, the present invention provides a method for detecting the presence of NOTCH3 mRNA in a sample comprising contacting the sample with at least one isolated oligonucleotide comprising the sequence of SEQ ID NO:35-43. The primer sets provided herein can be used for quantitating NOTCH3 mRNA levels in a sample following standard qRT-PCR procedures.

In one embodiment of the invention, microarrays are used to determine NOTCH3 mRNA levels in biological samples. Microarrays are particularly well suited for this purpose because of their reproducibility. DNA microarrays provide one method for the simultaneous measurement of the expression levels of large numbers of genes or a large number of oligonucleotide probes directed to different parts of a molecule of interest. Each array consists of a reproducible pattern of capture probes attached to a solid support. Labeled RNA or DNA is hybridized to complementary probes on the array and then detected by for example, laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNAs in a sample.

Techniques for the synthesis of these arrays using mechanical synthesis methods are described in, e.g., U.S. Pat. No. 5,384,261, incorporated herein by reference in its entirety. Although a planar array surface is preferred, the array can be fabricated on a surface of virtually any shape or even a multiplicity of surfaces. Arrays can be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety. Arrays can be packaged in such a manner as to allow for diagnostics or other manipulation of an all-inclusive device. See, for example, U.S. Pat. Nos. 5,856,174 and 5,922,591 herein incorporated by reference.

Methods for detecting the level of NOTCH3 protein its tumor cells can comprise any method that detects the presence of NOTCH3 protein in a biological sample. Such methods are well known in the art and include, but are not limited to, Western blots, slot blots, ELISA, immunoprecipitation, immunofluorescence, flow cytometry, immunocytochemistry, immunohistochemistry (IHC), and mass spectroscopy. Such immunoassay methods can be performed manually or in an automated fashion. Antibodies that bind any region of NOTCH3 are useful in the detection methods described herein. In one embodiment, the level of NOTCH3 protein in a tumor sample is determined using IHC.

Techniques for detecting antibody binding are well known in the art. Antibody binding to NOTCH3 protein can be detected through the use of chemical reagents that generate a detectable signal that corresponds to the level of antibody binding and, accordingly, to the level of NOTCH3 protein. In one embodiment, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the mutation of interest. Enzymes of particular interest include horseradish peroxidase (HRP) and alkaline phosphatase (AP). Commercial antibody detection systems, such as, for example the Dako Envision+ system (Dako North America, Inc., Carpinteria, Calif.) and Mach 3 system (Biocare Medical, Walnut Creek, Calif.), can be used to practice the present invention.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrim; an example of a luminescent material includes luminol; examples of bioluminescent materials include luciferase, luciferin, and aequorin; and examples of suitable radioactive material include ¹²⁵I, ¹³¹I, ³⁵S, or ³H.

In one embodiment, the level of NOTCH3 protein is determined using an agent that specifically binds to NOTCH3. Any molecular entity that displays specific binding to NOTCH3 can be employed to determine the level of NOTCH3 protein in a sample. Specific binding agents include, but are not limited to, antibodies, antibody mimetics, and polynucleotides (e.g., aptamers). One of skill understands that the degree of specificity required is determined by the particular assay used to detect NOTCH3 protein. For example, an agent that specifically binds to both fall length NOTCH3 and NOTCH3 ICD can be used in a method that involves the separation of polypeptides based on their size, e.g. Western blot.

In one embodiment, the level of NOTCH3 protein is determined using an antibody specific for NOTCH3. In another embodiment, the antibody is a monoclonal antibody. NOTCH3 specific antibodies can be generated according to any method known to one of skill in the art. See, e.g., Tagami et al., 2008 Mol. Cell. Biol., 28(1):165-176. NOTCH3 specific antibodies are also available from commercial sources. See, e.g., R&D Systems, Anti-Human NOTCH3 Polyclonal Antibody, Catalog #BAF1559. The anti-NOTCH3 antibody can be monoclonal antibody, polyclonal antibody, humanized antibody, human antibody, chimeric antibody or an antigen binding fragment thereof. In a further embodiment, the antibody specifically binds to NOTCH3 in a fixed and embedded tissue sample. The tissue sample can be a formalin fixed tissue sample. The tissue sample can be a paraffin embedded tissue sample.

### 3. NOTCH Inhibitors

Another aspect of the methods of the invention is the use of a NOTCH inhibitor (e.g., anti-NOTCH antibody) for treating pancreatic cancer patients whose NOTCH3 expression levels have been determined. In certain embodiments, the NOTCH inhibitor is an anti-NOTCH antibody. In certain embodiments, the anti-NOTCH antibody specifically binds to an EGF10 domain (or an equivalent of an EGF 10 domain) of one or more human NOTCH receptors. In certain embodiments, the anti-NOTCH antibody specifically binds to EGF 10 of human NOTCH2 and/or EGF9 of human NOTCH3. EGF9 is the EGF within human NOTCH3 that is equivalent to EGF10 in the other human NOTCH receptors NOTCH1, NOTCH2, and NOTCH4. In some embodiments, the anti-NOTCH antibody specifically binds to EGF10 of NOTCH2. In some embodiments, the anti-NOTCH antibody specifically binds to EGF10 of NOTCH2 and to EGF9 of NOTCH3. In some embodiments, the anti-NOTCH antibody specifically binds to EGF9 of NOTCH3. In other embodiments, the anti-NOTCH antibody binds to at least part of the sequence HKGAL (SEQ ID NO:1) within NOTCH2 EGF10. In some embodiments, the anti-NOTCH antibody binds to at least part of the sequence HEDAI (SEQ ID NO:2) within NOTCH3 EGF9. Exemplary antibodies that bind NOTCH2 and NOTCH3 are described in U.S. Pat. No. 8,226,943, which is incorporated herein by reference in its entirety.

In certain embodiments, an anti-NOTCH antibody that is useful in the methods of the invention inhibits binding of a ligand to human NOTCH2 and/or NOTCH3. In some embodiments, the anti-NOTCH antibody inhibits binding of a ligand to human NOTCH2. In some embodiments, the anti-NOTCH antibody inhibits binding of a ligand to NOTCH2 and NOTCH3. In other embodiments, the anti-NOTCH antibody inhibits binding of a ligand to NOTCH3. In certain embodiments, the ligand is DLL4, JAG1 or JAG2. In other embodiments, the anti-NOTCH antibody inhibits signaling of human NOTCH2 and/or NOTCH3. In some embodiments, the anti-NOTCH antibody inhibits signaling of human NOTCH2. In some embodiments, the anti-NOTCH antibody inhibits signaling of NOTCH2 and NOTCH3. In other embodiments, the anti-NOTCH antibody inhibits signaling of NOTCH3. In some embodiments NOTCH2 and/or NOTCH3 signaling is induced by DLL4, JAG1 or JAG2.

In certain embodiments, an anti-NOTCH antibody that is useful in the methods of the invention specifically binds human NOTCH2 and/or NOTCH3, wherein the antibody comprises (a) a heavy chain CDR1 comprising SSSGMS (SEQ ID NO:3), a heavy chain CDR2 comprising VIASSGSNTYYADSVKG (SEQ ID NO:4), and/or a heavy chain CDR3 comprising SIFYTT (SEQ ID NO:9); and/or (b) a light chain CDR1 comprising RASQSVRSNYLA (SEQ ID NO:6), a light chain CDR2 comprising GASSRAT (SEQ ID NO:7), and/or a light chain CDR3 comprising QQYSNFPI (SEQ ID NO:8). In some embodiments, the antibody comprises (a) a heavy chain CDR1 comprising SSSGMS (SEQ ID NO:3), or a variant thereof comprising 1, 2, 3, or 4 conservative amino acid substitutions; a heavy chain CDR2 comprising VIASSGSNTYYADSVKG (SEQ ID NO:4), or a variant thereof comprising 1, 2, 3, or 4 conservative amino acid substitutions; and/or a heavy chain CDR3 comprising SIFYTT (SEQ ID NO:9), or a variant thereof comprising 1, 2, 3, or 4 conservative amino acid substitutions; and/or (b) a light chain CDR1 comprising RASQSVRSNYLA (SEQ ID NO:6), or a variant thereof comprising 1, 2, 3, or 4 conservative amino acid substitutions; a light chain CDR2 comprising GASSRAT (SEQ ID NO:7), or a variant thereof comprising 1, 2, 3, or 4 conservative amino acid substitutions; and/or a light chain CDR3 comprising QQYSNFPI (SEQ ID NO:8), or a variant thereof comprising 1, 2, 3, or 4 conservative amino acid substitutions.

In certain embodiments, an anti-NOTCH antibody that is useful in the methods of the invention specifically binds human NOTCH2 and/or NOTCH3, wherein the antibody comprises (a) a heavy chain CDR1 comprising SSSGMS (SEQ ID NO:3), a heavy chain CDR2 comprising VIASSGSNTYYADSVKG (SEQ ID NO:4), and/or a heavy chain CDR3 comprising GIFFAI (SEQ ID NO:5); and/or (b) a light chain CDR1 comprising RASQSVRSNYLA (SEQ ID NO:6), a light chain CDR2 comprising GASSRAT (SEQ ID NO:7), and/or a light chain CDR3 comprising QQYSNFPI (SEQ ID NO:8). In certain embodiments, the antibody specifically binds NOTCH2. In some embodiments, the antibody comprises (a) a heavy chain CDR1 comprising SSSGMS (SEQ ID NO:3), or a variant thereof comprising 1, 2, 3, or 4 conservative amino acid substitutions; a heavy chain CDR2 comprising VIASSGSNTYYADSVKG (SEQ ID NO:4), or a variant thereof comprising 1, 2, 3, or 4 conservative amino acid substitutions; and/or a heavy chain CDR3 comprising GIFFAI (SEQ ID NO:5), or a variant thereof comprising 1, 2, 3, or 4 conservative amino acid substitutions; and/or (b) a light chain CDR1 comprising RASQSVRSNYLA (SEQ ID NO:6), or a variant thereof comprising 1, 2, 3, or 4 conservative amino acid substitutions; a light chain CDR2 comprising GASSRAT (SEQ ID NO:7), or a variant thereof comprising 1, 2, 3, or 4 conservative amino acid substitutions; and/or a light chain CDR3 comprising QQYSNFPI (SEQ ID NO:8), or a variant thereof comprising 1, 2, 3, or 4 conservative amino acid substitutions.

In certain embodiments, an anti-NOTCH antibody that is useful in the methods of the invention specifically binds human NOTCH2 and/or NOTCH3, wherein the antibody comprises (a) a heavy chain CDR1 comprising SSSGMS (SEQ ID NO:3), a heavy chain CDR2 comprising VIASSGSNTYYADSVKG (SEQ ID NO:4), and/or a heavy chain CDR3 comprising (G/S)(I/S)F(F/Y)(A/P)(I/T/S/N) (SEQ ID NO:10); and/or (b) a light chain CDR1 comprising RASQSVRSNYLA (SEQ ID NO:6), a light chain CDR2 comprising GASSRAT (SEQ ID NO:7), and/or a light chain CDR3 comprising QQYSNFPI (SEQ ID NO:8). In some embodiments, the antibody comprises a heavy chain CDR3 comprising SIFYPT (SEQ ID NO:11). In some embodiments, the antibody comprises a heavy chain CDR3 comprising SSSFFAS (SEQ ID NO:12). In other embodiments, the antibody comprises a heavy chain CDR3 comprising SSFYAS (SEQ ID NO:13). In certain embodiments, the antibody comprises a heavy chain CDR3 comprising SSFFAT (SEQ ID NO:14). In some embodiments, the antibody comprises a heavy chain CDR3 comprising SIFYPS (SEQ ID NO:15). In yet other embodiments, the antibody comprises a heavy chain CDR3 comprising SSFFAN (SEQ ID NO:16).

In certain embodiments, an anti-NOTCH antibody that is useful in the methods of the invention comprises: (a) a heavy chain variable region having at least about 80% sequence identity to SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26 (with or without signal sequence); and/or (b) a light chain variable region having at least about 80% sequence identity to SEQ ID NO:29, SEQ ID NO:27 or SEQ ID NO:28 (with or without signal sequence). In certain embodiments, the anti-NOTCH antibody specifically binds human NOTCH2 and/or NOTCH3. In some embodiments, the anti-NOTCH antibody specifically binds to human NOTCH2. In some embodiments, the anti-NOTCH antibody binds to NOTCH2 and NOTCH3. In other embodiments, the anti-NOTCH antibody binds to NOTCH3. In certain embodiments, the anti-NOTCH antibody comprises a heavy chain variable region having at least about 85%, at least about 90%, at least about 95%, at least about 98%, or about 100% sequence identity to SEQ ID NO:18 or SEQ ID NO:17. In certain embodiments, the anti-NOTCH antibody comprises a light chain variable region having at least about 85%, at least about 90%, at least about 95%, at least about 98%, or about 100% sequence identity to SEQ ID NO:29.

In certain embodiments, an anti-NOTCH antibody that is useful in the methods of the invention comprises: (a) a heavy chain having at least about 80% sequence identity to SEQ ID NO:30, SEQ ID NO:31, or SEQ ID NO:32 (with or without signal sequence); and/or (b) a light chain having at least about 80% sequence identity to SEQ ID NO:33, or SEQ ID NO:34 (with or without signal sequence). In certain embodiments, the anti-NOTCH antibody comprises a heavy chain having at least about 85%, at least about 90%, at least about 95%, at least about 98%, or about 100% sequence identity to SEQ ID NO:19, and a light chain having at least about 85%, at least about 90%, at least about 95%, at least about 98%, or about 100% sequence identity to SEQ ID NO:28. In certain embodiments, the anti-NOTCH antibody comprises a heavy chain having at least about 85%, at least about 90%, at least about 95%, at least about 98%, or about 100% sequence identity to SEQ ID NO:30, and a light chain having at least about 85%, at least about 90%, at least about 95%, at least about 98%, or about 100% sequence identity to SEQ ID NO:28.

In certain embodiments, an anti-NOTCH antibody that is useful in the methods of the invention comprises: (a) a heavy chain variable region having at least about 80% sequence identity to SEQ ID NO:17; and/or (b) a light chain variable region having at least about 80% sequence identity to SEQ ID NO:29. In certain embodiments, the anti-NOTCH antibody comprises a heavy chain variable region having at least about 85%, at least about 90%, at least about 95%, at least about 98%, or about 100% sequence identity to SEQ ID NO:17, and a light chain variable region having at least about 85%, at least about 90%, at least about 95%, at least about 98%, or about 100% sequence identity to SEQ ID NO:29.

In certain embodiments, an anti-NOTCH antibody that is useful in the methods of the invention comprises, consists, or consists essentially of a 59R1 IgG2 antibody comprising the heavy chain and light chain of SEQ ID NOs:31 and 33 (with or without signal sequence), respectively, or as encoded by the DNA deposited with the American Type Culture Collection (ATCC), 10801 University Boulevard, Manassas, Va., , under the conditions of the Budapest Treaty on Oct. 15, 2008, and assigned designation number PTA-9547.

In certain embodiments, an anti-NOTCH antibody that is useful in the methods of the invention comprises, consists or consists essentially of a 59R5 IgG2 antibody comprising the heavy chain and light chain of SEQ ID NO:30 and SEQ ID NO:33 (with or without signal sequence), respectively, or as encoded by the DNA deposited with the ATCC on Jul. 6, 2009, and assigned designation number PTA-10170. In certain embodiments, the anti-NOTCH antibody useful in the methods of the invention comprises the heavy chains and light chains of the 59R5 IgG2 antibody (with or without the leader sequence). In certain embodiments, the anti-NOTCH antibody that is useful in the methods of the invention is the 59R5 IgG2 antibody. The 59R5 IgG2 antibody is also referred to herein as OMP-59R5. Additional information regarding the OMP-59R5 antibody can be found, for example, in U.S. Pat. No. 8,226,943, which is incorporated by reference herein in its entirety. In U.S. Pat. No. 8,226,943, the OMP-59R5 antibody is generally referred to as “59R5” or the “59R5 IgG2 antibody.”

In certain embodiments, an anti-NOTCH antibody that is useful in the methods of the invention competes for specific binding to human NOTCH2 and/or NOTCH3 with an antibody comprising a heavy chain variable region comprising SEQ ID NO:18 and a light chain variable region comprising SEQ ID NO:29. In certain embodiments, the antibody competes for specific binding with a 59R1 IgG2 antibody comprising the heavy chain and light chain of SEQ ID NOs:31 and 33 (with or without signal sequence), respectively, or as encoded by the DNA deposited with the ATCC on Oct. 15, 2008, and assigned designation number PTA-9547. In some embodiments, the antibody competes for binding to human NOTCH2. In some embodiments, the antibody competes for binding to human NOTCH2 and NOTCH3. In other embodiments, the antibody competes for binding to human NOTCH3.

In certain embodiments, an anti-NOTCH antibody that is useful in the methods of the invention competes for specific binding to human NOTCH2 and/or NOTCH3 with an antibody comprising a heavy chain variable region comprising SEQ ID NO:17 and a light chain variable region comprising SEQ ID NO:29. In some embodiments, the antibody competes for specific binding with a 59R5 antibody comprising the heavy chain and light chain of SEQ ID NOs:30 and 33, respectively, or as encoded by the DNA deposited with the ATCC on Jul. 6, 2009, and assigned designation number PTA-10170. In some embodiments, the antibody competes for binding to human NOTCH2. In some embodiments, the antibody competes for binding to human NOTCH2 and NOTCH3. In other embodiments, the antibody competes for binding to human NOTCH3.

In certain embodiments, an anti-NOTCH antibody that is useful in the methods of the invention is an IgG1 antibody or an IgG2 antibody. In certain embodiments, the antibody is a monoclonal antibody. In certain embodiments, the antibody is a human antibody or a humanized antibody. In certain embodiments, the antibody is an antibody fragment.

In certain embodiments, an anti-NOTCH antibody that is useful in the methods of the invention binds to the same epitope as or binds to an epitope that overlaps with the epitope of the 59R1 or 59R5 antibody.

Further examples of anti-NOTCH antibodies useful in the methods of the invention are disclosed in U.S. Pat. No. 8,226,943, which is incorporated by reference herein in its entirety.

In certain embodiments, an anti-NOTCH antibody that is useful in the methods of the invention is a bispecific antibody that specifically recognizes a human NOTCH receptor. Bispecific antibodies are antibodies that are capable of specifically recognizing and binding at least two different epitopes. In one embodiment, the bispecific anti-NOTCH antibody specifically recognizes different epitopes within the same human NOTCH receptor. In another embodiment, the bispecific anti-NOTCH antibody specifically recognizes different epitopes within a human NOTCH receptor or on different human NOTCH receptors.

Alternatively, in certain alternative embodiments, an anti-NOTCH antibody that is useful in the methods of the invention is not a bispecific antibody.

In certain embodiments, an anti-NOTCH antibody that is useful in the methods of the invention is monospecific. For example, in certain embodiments, each of the one or more antigen-binding sites that an antibody contains is capable of binding (or binds) the same one or more human NOTCH receptors. In certain embodiments, an antigen-binding site of the monospecific anti-NOTCH antibody is capable of binding (or binds) one, two, three, or four human NOTCH receptors.

Another aspect of the methods of the invention is the use of a NOTCH inhibitor (e.g., anti-NOTCH antibody) in the treatment of pancreatic cancer. In certain embodiments, the NOTCH inhibitors are inhibitors for gamma-secretase. Because gamma-secretase inhibitors are also able to prevent NOTCH receptor activation, several forms of gamma-secretase inhibitors have been tested for antitumor effects. First, an original gamma-secretase inhibitor, IL-X (cbz-IL-CHO), was shown to have NOTCH1-dependent antineoplastic activity in Ras-transformed fibroblasts. A tripeptide gamma-secretase inhibitor (z-Leu-leu-Nle-CHO) was reported to suppress tumor growth in cell lines and/or xenografts in mice from melanoma and Kaposi sarcoma (Curry C L et al., Oncogene 24:6333-44(2005)). Treatment with dipeptide gamma-secretase inhibitor N-[N-(3,5-difluorophenacetyl)-L-alanyl]-S-phenylglycine t-butyl ester (DAPT) also resulted in a marked reduction in medulloblastoma growth and induced G0-G1 cell cycle arrest and apoptosis in a T-ALL animal model (O'Neil J. et al., Blood 107:781-5 (2006)). Another gamma-secretase inhibitor, dibenzazepine, has been shown to inhibit epithelial cell proliferation and induce goblet cell differentiation in intestinal adenomas in Apc−/− (min) mice (van Es J H, et al., Nature 435:959-63 (2005)). More recently, functional inactivation of NOTCH3 either by tripeptide gamma-secretase inhibitor or NOTCH3-specific small interfering RNA results in suppression of cell proliferation and induction of apoptosis in the tumor cell lines that overexpressed NOTCH3 but not in those with minimal amounts of NOTCH3 expression (Park J T et al., Cancer Res. 66: 6312-8 (2006)). Furthermore, a phase I clinical trial for a NOTCH inhibitor, MK0752 (developed by Merck, Whitehouse Station, N.J.), has been launched for relapsed or refractory T-ALL patients and advanced breast cancers.

### 4. Methods of Treatment

As described above, NOTCH inhibitors (e.g., OMP-59R5) can be used to treat pancreatic cancer in a patient whose tumor cells have been determined to possess increased levels of NOTCH3 expression (e.g., NOTCH3 mRNA expression), e.g., levels at or above the median level for NOTCH3 expression in pancreatic cancers, levels at or above the 95^{th}, 90^{th}, 80^{th}, 75^{th}, 70^{th}, 50^{th}, 40^{th}, 30^{th}, 25^{th} or 10^{th} percentile for NOTCH3 expression of pancreatic cancers, or levels at or above the level of NOTCH3 expression of a control sample. In certain embodiments, the tumor cells have also been determined to possess increased levels of MAML2 expression (e.g., MAML2 mRNA expression), for example, levels at or above the median level for MAML2 expression in pancreatic cancers, or levels at or above the level of MAML2 expression of a control sample. In certain embodiments, the NOTCH inhibitors (e.g., OMP-59R5) are useful in inhibiting tumor growth, inducing differentiation, and/or r educing tumor volume. In addition, the invention provides a method of reducing the tumorigenicity of a pancreatic tumor in a subject, comprising administering a therapeutically effective amount of a NOTCH inhibitor (e.g., OMP-59R5) to a patient whose tumor cells have been determined to express increased levels of NOTCH3 as described herein. In certain embodiments, the tumor comprises cancer stem cells. In certain embodiments, the frequency of cancer stem cells in the tumor is reduced by administration of the NOTCH inhibitor (e.g., OMP-59R5).

In one embodiment, NOTCH inhibitors (e.g., OMP-59R5) can be used to treat a pancreatic cancer whose tumor cells are characterized by having a level of NOTCH3 expression at or above the level of NOTCH3 expression in a control sample or cell. In one embodiment, NOTCH inhibitors (e.g., OMP-59R5) can be used to treat a pancreatic cancer whose tumor cells are characterized by having a level of NOTCH3 gene expression at or above the median level of NOTCH3 expression of pancreatic cancers. In certain embodiments, the pancreatic cancer treated comprises tumor cells characterized by having a level of NOTCH3 expression at or above the 95^{th}, 90^{th}, 80^{th}, 75^{th}, 70^{th}, 50^{th}, 40^{th}, 30^{th}, 25^{th} or 10^{th} percentile for NOTCH3 expression in pancreatic cancers. In certain embodiments, the median level of NOTCH3 expression of pancreatic cancers is the median level of NOTCH3 expression of pancreatic adenocarcinomas, metastatic pancreatic cancers, or liver and/or lymph node metastatic pancreatic cancers. In certain embodiments, the 95^{th}, 90^{th}, 80^{th}, 75^{th}, 70^{th}, 50^{th}, 40^{th}, 30^{th}, 25^{th} or 10^{th} percentile for NOTCH3 expression in pancreatic cancers is the 95^{th}, 90^{th}, 80^{th}, 75^{th}, 70^{th}, 50^{th}, 40^{th}, 30^{th}, 25^{th} or 10^{th} percentile for NOTCH3 expression in pancreatic adenocarcinomas, metastatic pancreatic cancers, or liver and/or lymph node metastatic pancreatic cancers. In certain embodiments, NOTCH3 expression level is determined using qRT-PCR. In certain embodiments, NOTCH3 expression level is determined using the probes described herein, for example, using a polynucleotide comprising a nucleotide sequence selected from the group consisting of SEQ ID NO:35-43.

In one embodiment, NOTCH inhibitors (e.g., OMP-59R5) can be used to treat a pancreatic cancer that comprises tumor cells at least some of which demonstrate a level of MAML2 expression at or above the level of MAML2 expression in a control cell. In one embodiment, NOTCH inhibitors (e.g., OMP-59R5) can be used to treat a pancreatic cancer that comprises tumor cells at least some of which demonstrate a level of MAML2 expression at or above the median level of MAML2 expression of pancreatic cancers. In certain embodiments, the pancreatic cancer treated comprises tumor cells at least some of which demonstrate a level of MAML2 expression at or above the 95^{th}, 90^{th}, 80^{th}, 75^{th}, 70^{th}, 50^{th}, 40^{th}, 30^{th}, 25^{th} or 10^{th} percentile for MAML2 expression in pancreatic cancers. In certain embodiments, the median level of MAML2 expression of pancreatic cancers is the median level of MAML2 expression of pancreatic adenocarcinomas, metastatic pancreatic cancers, or liver and/or lymph node metastatic pancreatic cancers. In certain embodiments, the 95^{th}, 90^{th}, 80^{th}, 75^{th}, 70^{th}, 50^{th}, 40^{th}, 30^{th}, 25^{th} or 10^{th} percentile for MAML2 expression in pancreatic cancers is the 95^{th}, 90^{th}, 80^{th}, 75^{th}, 70^{th}, 50^{th}, 40^{th}, 30^{th}, 25^{th} or 10^{th} percentile for MAML2 expression in pancreatic adenocarcinomas, metastatic pancreatic cancers, or liver and/or lymph node metastatic pancreatic cancers. In certain embodiments, MAML2 expression level is determined using qRT-PCR.

In certain embodiments, the pancreatic cancer that is treated with a NOTCH inhibitor (e.g., OMP-59R5) is an exocrine tumor of the pancreas. In certain embodiments, the pancreatic cancer treated is acinar cell carcinoma, adenocarcinoma, adenosquamous carcinoma, giant cell tumor, intraductal papillary-mucinous neoplasm (IPMN), mucinous cystadenocarcinoma, pancreatoblastoma, serous cystadenocarcinoma, or solid and pseudopapillary tumor. In certain embodiments, the pancreatic cancer treated is adenocarcinoma. In certain embodiments, the pancreatic cancer treated is a neuroendocrine tumor. In certain embodiments, the pancreatic neuroendocrine tumor is a gastrinoma, glucagonoma, insulinoma, nonfunctional islet cell tumor, VIPoma or somatostatinoma. In certain embodiments, the pancreatic cancer treated is not a neuroendocrine tumor.

In certain embodiments, the pancreatic cancer that is treated with a NOTCH inhibitor (e.g., OMP-59R5) is resectable tumor, locally advanced cancer, or metastatic pancreatic cancer. In certain embodiments, the pancreatic cancer is a grade 1, 2, 3 or 4 cancer as determined according to the AJCC TNM system.

In one embodiment, the NOTCH inhibitors (e.g., OMP-59R5) are particularly useful in treating pancreatic cancer patients that have already undergone some form of treatment. In another embodiment, the NOTCH inhibitors (e.g., OMP-59R5) are used to treat a pancreatic cancer patient that previously failed with a cancer therapy. Failed cancer therapies can include, but are not limited to, chemotherapy, adjuvant therapy, neoadjuvant therapy, and combinations thereof. In one embodiment, the NOTCH inhibitors (e.g., OMP-59R5) are used to treat chemotherapy resistant tumors. In another embodiment, the NOTCH inhibitors (e.g., OMP-59R5) are used to treat chemotherapy resistant pancreatic cancer.

In one embodiment, the treatment method involves first testing a biological sample containing pancreatic cancer cells from a patient to determine whether they express the NOTCH3 gene at or above a predetermined standard, e.g., at or above the median level for NOTCH3 expression in pancreatic cancer. Patients whose samples demonstrate elevated level of NOTCH3 expression would then be treated using a NOTCH inhibitor (e.g., OMP-59R5) that interferes with NOTCH receptor activity. The dosage administered will depend upon the particular condition being treated, the route of administration and clinical considerations that are well known in the art. Dosages can be gradually increased until a beneficial effect, e.g., a slowing of tumor growth, is detected. The NOTCH inhibitors (e.g., OMP-59R5) can then be provided in either single or multiple dosage regimens and can be given either alone or in conjunction with other therapeutic agents.

Treatment of pancreatic cancers with increased NOTCH3 expression is compatible with any route of administration and dosage form. Depending upon the particular condition being treated, certain dosage forms will tend to be more convenient or effective than others. For example, NOTCH inhibitors can be administered parenterally, topically, orally, perorally, internally, intranasally, rectally, vaginally, lingually and transdermally. Specific dosage forms include tablets, pills, capsules, powders, aerosols, suppositories, skin patches, parenterals and oral liquids including suspensions, solutions and emulsions. Sustained release dosage forms can also be used. All dosage forms can be prepared using methods that are standard in the art (see, e.g., Remington's Pharmaceutical Sciences, 16th ed., Easton, Pa. (1980)).

In certain embodiments, the administration of a NOTCH inhibitor (e.g., OMP-59R5) can be by intravenous injection or intravenously. In some embodiments, the administration is by intravenous infusion. In some embodiments, the administration of the NOTCH inhibitor (e.g., OMP-59R5) can be by a non-intravenous route.

The appropriate dosage of a NOTCH inhibitor (e.g., OMP-59R5) therapeutic agent depends on the severity and course of the disease, the responsiveness of the disease, whether the antibody or NOTCH inhibitor is administered for therapeutic or preventative purposes, previous therapy, patient's clinical history, and so on all at the discretion of the treating physician. The antibody or other NOTCH inhibitor can be administered one time or over a series of treatments lasting from several days to several months, or until a cure is effected or a diminution of the disease state is achieved (e.g. reduction in tumor size). Optimal dosing schedules can be calculated from measurements of drug accumulation in the body of the patient and will vary depending on the relative potency of an individual antibody or other NOTCH inhibitor. The administering physician can easily determine optimum dosages, dosing methodologies and repetition rates. In general, dosage of an anti-NOTCH antibody (e.g., OMP-59R5) is from 0.01 μg to 100 mg per kg of body weight, and can be given once or more daily, weekly, monthly or yearly. The treating physician can estimate repetition rates for dosing based on measured residence times and concentrations of the antibody or agent in bodily fluids or tissues.

As is known by those of skill in the art, doses used will vary depending on the clinical goals to be achieved. In some embodiments, each dose of the anti-NOTCH antibody (e.g., OMP-59R5) is about 0.25 mg/kg to about 15 mg/kg. In some embodiments, each dose is about 0.25, 0.5, 1, 2, 3, 4, 5, 6, 7, 8, 9, 10, 11, 12, 13, 14, 15, 16, 17, 18, 19, or 20 mg/kg. In certain embodiments, each dose is about 0.5 mg/kg. In certain embodiments, each dose is about 1 mg/kg. In certain embodiments, each dose is about 2.5 mg/kg. In certain embodiments, each dose is about 5 mg/kg. In certain embodiments, each dose is about 7.5 mg/kg. In certain embodiments, each dose is about 10mg/kg. In certain embodiments, each dose is about 12.5 mg/kg. In certain embodiments, each dose is about 15 mg/kg.

In certain embodiments, the NOTCH inhibitor (e.g., OMP-59R5) used in the methods described herein is administered to the patient using an intermittent dosing regimen, which may in some instances reduce side effects and/or toxicities associated with administration of the NOTCH inhibitor (e.g., OMP-59R5). As used herein, “intermittent dosing” refers to a dosing regimen using a dosing interval of more than once a week, e.g., dosing once every 2 weeks, once every 3 weeks, once every 4 weeks, etc. In some embodiments, a method for treating pancreatic cancer in a human patient comprises administering to the patient an effective dose of a NOTCH inhibitor (e.g., OMP-59R5) according to an intermittent dosing regimen. In some embodiments, a method for treating pancreatic cancer in a human patient comprises administering to the patient an effective dose of a NOTCH inhibitor (e.g., OMP-59R5) according to an intermittent dosing regimen, and increasing the therapeutic index of the NOTCH inhibitor (e.g., OMP-59R5). In some embodiments, the intermittent dosing regimen comprises administering an initial dose of a NOTCH inhibitor (e.g., OMP-59R5) to the patient, and administering subsequent doses of the NOTCH inhibitor (e.g., OMP-59R5) about once every 2 weeks. In some embodiments, the intermittent dosing regimen comprises administering an initial dose of a NOTCH inhibitor (e.g., OMP-59R5) to the patient, and administering subsequent doses of the NOTCH inhibitor (e.g., OMP-59R5) about once every 3 weeks. In some embodiments, the intermittent dosing regimen comprises administering an initial dose of a NOTCH inhibitor (e.g., OMP-59R5) to the patient, and administering subsequent doses of the NOTCH inhibitor (e.g., OMP-59R5) about once every 4 weeks.

In some alternative embodiments, the anti-NOTCH antibody used in the methods is OMP-59R5, or an antibody comprising the six CDRs and/or the variable regions of OMP-59R5, and the antibody is administered to subjects intravenously at a dosage of about 2.5 mg/kg to about 7.5 mg/kg (e.g., about 2.5 mg/kg, about 5 mg/kg, or about 7.5 mg/kg) approximately every two to three weeks.

In certain embodiments, in addition to administering a NOTCH inhibitor (e.g., OMP-59R5), the method or treatment further comprises administering at least one additional therapeutic agent or therapy. An additional therapeutic agent or therapy can be administered prior to, concurrently with, and/or subsequently to, administration of the anti-NOTCH therapeutic agent. In some embodiments, the at least one additional therapeutic agent or therapy comprises 1, 2, 3, or more additional therapeutic agents or therapies.

Combination therapy with at least two therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects. Combination therapy may decrease the likelihood that resistant cancer cells will develop.

It will be appreciated that the combination of a NOTCH inhibitor (e.g., OMP-59R5) and an additional therapeutic agent or therapy can be administered in any order or concurrently. In some embodiments, the NOTCH inhibitor (e.g., OMP-59R5) will be administered to patients that have previously undergone treatment with a second therapeutic agent or therapy. In certain other embodiments, the NOTCH inhibitor (e.g., OMP-59R5) and a second therapeutic agent or therapy will be administered substantially simultaneously or concurrently. For example, a subject can be given the NOTCH inhibitor (e.g., OMP-59R5) agent while undergoing a course of treatment with a second therapeutic agent (e.g., chemotherapy). In certain embodiments, the NOTCH inhibitor (e.g., OMP-59R5) will be administered within 1 year of the treatment with a second therapeutic agent. In certain alternative embodiments, the NOTCH inhibitor (e.g., OMP-59R5) will be administered within 10, 8, 6, 4, or 2 months of any treatment with a second therapeutic agent. In certain other embodiments, the NOTCH inhibitor (e.g., OMP-59R5) will be administered within 4, 3, 2, or 1 weeks of any treatment with a second therapeutic agent. In some embodiments, the NOTCH inhibitor (e.g., OMP-59R5) will be administered within 5, 4, 3, 2, or 1 days of any treatment with a second therapeutic agent. It will further be appreciated that the two (or more) agents or treatments can be administered to the subject within a matter of hours or minutes (i.e., substantially simultaneously).

As is known by those of skill in the art, doses used will vary depending on the clinical goals to be achieved. In some embodiments, each dose of an anti-NOTCH antibody (e.g., OMP-59R5) is about 0.25 mg/kg to about 15 mg/kg. In some embodiments, each dose is about 0.25, 0.5, 1, 2, 3, 4, 5, 6, 7, 8, 9, 10, 11, 12, 13, 14, 15, 16, 17, 18, 19, or 20 mg/kg. In certain embodiments, each dose is about 0.5 mg/kg. In certain embodiments, each dose is about 1 mg/kg. In certain embodiments, each dose is about 2.5 mg/kg. In certain embodiments, each dose is about 5 mg/kg. In certain embodiments, each dose is about 7.5 mg/kg. In certain embodiments, each dose is about 10 mg/kg. In certain embodiments, each dose is about 12.5 mg/kg. In certain embodiments, each dose is about 15 mg/kg.

In certain embodiments, a method treating pancreatic cancer described herein comprises the administration of a NOTCH inhibitor (e.g., OMP-59R5) in combination with one or more chemotherapeutic agents. Thus, in some embodiments, the method or treatment involves the combined administration of a NOTCH inhibitor (e.g., OMP-59R5) and a chemotherapeutic agent or cocktail of multiple different chemotherapeutic agents. In certain embodiments, a method described herein comprises administering to a pancreatic cancer patient a therapeutically effective amount of the OMP-59R5 antibody in combination with gemcitabine and ABRAXANE™ (protein bound paclitaxel). Treatment with a NOTCH inhibitor (e.g., OMP-59R5) can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in Chemotherapy Service Editor M. C. Perry, Williams & Wilkins, Baltimore, Md. (1992).

Chemotherapeutic agents useful in the instant invention include, but are not limited to, alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofaran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel and docetaxel; chlorambucil; gemcitabine; 6-thioguanine; mercaptopurine; platinum analogs such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine; retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide, and irinotecan, as well as pharmaceutically acceptable salts, acids, or derivatives of any of these.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, a method described herein comprises administering to a pancreatic cancer patient a therapeutically effective amount of the OMP-59R5 antibody in combination with an anti-metabolite. In certain embodiments, the anti-metabolite is a nucleoside analogue. In certain embodiments, a method described herein comprises administering to a pancreatic cancer patient a therapeutically effective amount of the OMP-59R5 antibody in combination with gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain alternative embodiments, the antimitotic agent comprises a vinca alkaloid, such as vincristine, binblastine, vinorelbine, or vindesine, or pharmaceutically acceptable salts, acids, or derivatives thereof. In certain embodiments, a method described herein comprises administering to a pancreatic cancer patient a therapeutically effective amount of the OMP-59R5 antibody in combination with an antimitotic agent. In certain embodiments, the anti-metabolite is a taxane. In certain embodiments, a method described herein comprises administering to a pancreatic cancer patient a therapeutically effective amount of the OMP-59R5 antibody in combination with ABRAXANE™ (protein bound paclitaxel).

In certain embodiments, the treatment involves the combined administration of an NOTCH inhibitor (e.g., OMP-59R5) and radiation therapy. Treatment with the NOTCH inhibitor (e.g., OMP-59R5) can occur prior to, concurrently with, or subsequent to administration of radiation therapy. Dosing schedules for such radiation therapy can be determined by the skilled medical practitioner. In some embodiments, the NOTCH inhibitor (e.g., OMP-59R5) is administered after radiation treatment. In some embodiments, the NOTCH inhibitor (e.g., OMP-59R5) is administered with radiation therapy.

In some embodiments, a second therapeutic agent comprises an antibody. Thus, treatment can involve the combined administration of an anti-NOTCH antibody (e.g., OMP-59R5) or other NOTCH inhibitor with other antibodies against additional tumor-associated antigens including, but not limited to, antibodies that bind to EGFR, ErbB2, DLL4, or NF-κB. Exemplary anti-DLL4 antibodies are described, for example, in U.S. Pat. No. 7,750,124. Additional anti-DLL4 antibodies are described in, e.g., International Patent Pub. Nos. WO 2008/091222 and WO 2008/0793326, and U.S. Patent Application Pub. Nos. 2008/0014196; 2008/0175847; 2008/0181899; and 2008/0107648. Combined administration can include co-administration, either in a single pharmaceutical formulation or using separate formulations, or consecutive administration in either order but generally within a time period such that all active agents can exert their biological activities simultaneously.

Furthermore, treatment with the NOTCH inhibitor (e.g., OMP-59R5) can include combination treatment with one or more cytokines (e.g., lymphokines, interleukins, tumor necrosis factors, and/or growth factors) or can be accompanied by surgical removal of tumors, cancer cells or any other therapy deemed necessary by a treating physician.

### 5. Antibodies and Production Thereof

Additional antibodies useful in the methods of the invention can be produced by any suitable method known in the art. Polyclonal antibodies can be prepared by any known method. Polyclonal antibodies are raised by immunizing an animal (e.g. a rabbit, rat, mouse, donkey, etc.) by multiple subcutaneous or intraperitoneal injections of the relevant antigen (a purified peptide fragment, full-length recombinant protein, fusion protein, etc.) optionally conjugated to keyhole limpet hemocyanin (KLH), serum albumin, etc. diluted in sterile saline and combined with an adjuvant (e.g. Complete or Incomplete Freund's Adjuvant) to form a stable emulsion. The polyclonal antibody is then recovered from blood, ascites and the like, of an animal so immunized. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc.

Monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g. radioimmunoassay (RIA); enzyme-linked immunosorbent assay (ELISA)) can then be propagated either in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

Alternatively monoclonal antibodies can also be made using recombinant DNA methods as described in U.S. Pat. No. 4,816,567. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries expressing CDRs of the desired species as described (McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, the monoclonal antibody useful in the methods of the invention is a humanized antibody. In certain embodiments, such antibodies are used therapeutically to reduce antigenicity and HAMA (human anti-mouse antibody) responses when administered to a human subject. Humanized antibodies can be produced using various techniques known in the art. In certain alternative embodiments, the antibody useful in the methods of the invention is a human antibody.

Human antibodies can be directly prepared using various techniques known in the art. Immortalized human B lymphocytes immunized in vitro or isolated from an immunized individual that produce an antibody directed against a target antigen can be generated (See, e.g., Cole et al., Monoclonal Antibodies and Cancer Therapy, Alan R. Liss, p. 77 (1985); Boemer et al., 1991, J. Immunol., 147 (1):86-95; and U.S. Pat. No. 5,750,373). Also, the human antibody can be selected from a phage library, where that phage library expresses human antibodies, as described, for example, in Vaughan et al., 1996, Nat. Biotech., 14:309-314, Sheets et al., 1998, Proc. Nat'l. Acad. Sci., 95:6157-6162, Hoogenboom and Winter, 1991, J. Mol. Biol., 227:381, and Marks et al., 1991, J. Mol. Biol., 222:581). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al., 2007, J. Mol. Bio., doi:10.1016/j.jmb.2007.12.018 (each of which is incorporated by reference in its entirety). Affinity maturation strategies and chain shuffling strategies (Marks et al., 1992, Bio/Technology 10:779-783, incorporated by reference in its entirety) are known in the art and can be employed to generate high affinity human antibodies.

Humanized antibodies can also be made in transgenic mice containing human immunoglobulin loci that are capable upon immunization of producing the fall repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In certain embodiments, the antibody useful in the methods of the invention is a bispecific antibody that specifically recognizes a human NOTCH receptor. Bispecific antibodies are antibodies that are capable of specifically recognizing and binding at least two different epitopes. The different epitopes can either be within the same molecule (e.g. the same human NOTCH receptor) or on different molecules. Bispecific antibodies can be intact antibodies or antibody fragments.

Alternatively, in certain alternative embodiments, antibodies useful for the invention are not bispecific antibodies.

In certain embodiments, the antibodies useful for the invention are monospecific. For example, in certain embodiments, each of the one or more antigen-binding sites that at an antibody contains is capable of binding (or binds) the same human NOTCH receptor. In certain embodiments, an antigen-binding site of a monospecific antibody is capable of binding (or binds) one, two, three, or four human NOTCH receptors.

In certain embodiments, an antibody useful for the methods of the invention is an antibody fragment. Antibody fragments can display increased tumor penetration relative to a full antibody. Various techniques are known for the production of antibody fragments. Traditionally, these fragments are derived via proteolytic digestion of intact antibodies (for example Morimoto et al., 1993, Journal of Biochemical and Biophysical Methods 24:107-117; Brennan et al., 1985, Science, 229:81). In certain embodiments, antibody fragments are produced recombinantly. Fab, Fv, and scFv antibody fragments can all be expressed in and secreted from E. coli or other host cells, thus allowing the production of large amounts of these fragments. Such antibody fragments can also be isolated from the antibody phage libraries discussed above. The antibody fragment can also be linear antibodies as described in U.S. Pat. No. 5,641,870, for example, and can be monospecific or bispecific. Single-chain antibodies useful in the methods of the invention can be prepared as described, for example, in U.S. Pat. No. 4,946,778. In addition, methods can be adapted for the construction of Fab expression libraries (Huse, et al., Science 246:1275-1281 (1989)) to allow rapid and effective identification of monoclonal Fab fragments with the desired specificity for a NOTCH receptor. Antibody fragments can be produced by techniques in the art including, but not limited to (a) a F(ab′)₂ fragment produced by pepsin digestion of an antibody molecule; (b) a Fab fragment generated by reducing the disulfide bridges of an F(ab′)₂ fragment, (c) a Fab fragment generated by the treatment of the antibody molecule with papain and a reducing agent, and (d) Fv fragments. Other techniques for the production of antibody fragments will be apparent to the skilled practitioner.

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

In certain embodiments, an antibody useful for the methods of the invention is a heteroconjugate antibody. Heteroconjugate antibodies are composed of two covalently joined antibodies. Such antibodies have, for example, been proposed to target immune cells to unwanted cells (U.S. Pat. No. 4,676,980). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

It is known in the art that the constant Fc region mediates several effector functions. For example, binding of the C1 component of complement to antibodies activates the complement system. Activation of complement is important in the opsonisation and lysis of cell pathogens. The activation of complement also stimulates the inflammatory response and can also be involved in autoimmune hypersensitivity. Further, antibodies or soluble receptors can bind to cells via the Fc region, with a Fc receptor site on the antibody Fc region binding to a Fc receptor (FcR) on a cell. There are a number of Fe receptors which are specific for different classes of antibody, including IgG (gamma receptors), IgE (epsilon receptors), IgA (alpha receptors) and IgM (mu receptors). Binding of antibody to Fe receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In certain embodiments, the NOTCH antagonist polypeptides (antibodies and Fc comprising soluble receptors) useful for the methods of the invention provide for altered effector functions that, in turn, affect the biological profile of the administered polypeptides. F or example, the deletion or inactivation (through point mutations or other means) of a constant region domain may reduce Fc receptor binding of the circulating modified antibody thereby increasing tumor localization. In other cases it may be that constant region modifications moderate complement binding and thus reduce the serum half-life and nonspecific association of a conjugated cytotoxin. Yet other modifications of the constant region may be used to eliminate disulfide linkages or oligosaccharide moieties that allow for enhanced localization due to increased antigen specificity or antibody flexibility. Similarly, modifications to the constant region can easily be made using well known biochemical or molecular engineering techniques well within the purview of the skilled artisan.

In certain embodiments, a NOTCH antagonist polypeptide comprising an Fc region (antibodies and Fc comprising soluble receptors) useful for the methods of the invention does not have one or more effector functions. For instance, in some embodiments, the polypeptide has no antibody-dependent cellular cytotoxicity (ADCC) activity and/or no complement-dependent cytotoxicity (CDC) activity. In certain embodiments, the polypeptide does not bind to an Fc receptor and/or complement factors. In certain embodiments, the antibody has no effector function.

The invention also pertains to the use of immunoconjugates comprising a NOTCH antagonist polypeptide (e.g., anti-NOTCH antibody) conjugated to a cytotoxic agent. Cytotoxic agents include chemotherapeutic agents, growth inhibitory agents, toxins (e.g., an enzymatically active toxin of bacterial, fungal, plant, or animal origin, or fragments thereof), radioactive isotopes (i.e., a radioconjugate), etc. Chemotherapeutic agents useful in the generation of such immunoconjugates include, for example, methotrexate, adriamicin, doxorubicin, melphalan, mitomycin C, chlorambucil, daunorubicin or other intercalating agents. Enzymatically active toxins and fragments thereof that can be used include diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies including ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). Conjugates of an antibody and one or more small molecule toxins, such as a calicheamicin, maytansinoids, a trichothene, and CC1065, and the derivatives of these toxins that have toxin activity, can also be used.

Conjugate antibodies are composed of two covalently joined antibodies. Such antibodies have, for example, been proposed to target immune cells to unwanted cells (U.S. Pat. No. 4,676,980). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

Regardless of how useful quantities are obtained, the NOTCH antagonists polypeptides (e.g., antibodies and soluble receptors) useful in the methods of the invention can be used in any one of a number of conjugated (i.e. an immunoconjugate) or unconjugated forms. Alternatively, the polypeptides can be used in a nonconjugated, or “naked” form. In certain embodiments, the polypeptides are used in nonconjugated form to harness the subject's natural defense mechanisms including complement-dependent cytotoxicity (CDC) and antibody dependent cellular toxicity (ADCC) to eliminate the malignant cells. In some embodiments, the polypeptides can be conjugated to radioisotopes, such as ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re using anyone of a number of well-known chelators or direct labeling. In other embodiments, the compositions can comprise NOTCH antagonist polypeptides coupled to drugs, prodrugs or biological response modifiers such as methotrexate, adriamycin, and lymphokines such as interferon. Still other embodiments comprise the use of NOTCH antagonist polypeptides conjugated to specific biotoxins such as ricin or diptheria toxin. In yet other embodiments the NOTCH antagonist polypeptides can be complexed with other immunologically active ligands (e.g. antibodies or fragments thereof) wherein the resulting molecule binds to both the neoplastic cell and an effector cell such as a T cell. The selection of which conjugated or unconjugated NOTCH antagonist polypeptides to use will depend of the type and stage of neuroendocrine tumor, use of adjunct treatment (e.g., chemotherapy or external radiation) and patient condition. It will be appreciated that one skilled in the art could readily make such a selection in view of the teachings herein.

The polypeptides and analogs can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in REMINGTON'S PHARMACEUTICAL SCIENCES, 20th ed., Mack Publishing Co., Easton, Pa. (2000).

The chemical moieties most suitable for derivatization include water soluble polymers. A water soluble polymer is desirable because the protein to which it is attached does not precipitate in an aqueous environment, such as a physiological environment. In some embodiments, the polymer will be pharmaceutically acceptable for the preparation of a therapeutic product or composition. One skilled in the art will be able to select the desired polymer based on such considerations as whether the polymer/protein conjugate will be used therapeutically, and if so, the desired dosage, circulation time, resistance to proteolysis, and other considerations. The effectiveness of the derivatization can be ascertained by administering the derivative, in the desired form (i.e., by osmotic pump, or by injection or infusion, or, further formulated for oral, pulmonary or other delivery routes), and determining its effectiveness. Suitable water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), dextran, poly(n-vinyl pyrrolidone)-polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde can have advantages in manufacturing due to its stability in water.

The isolated polypeptides (e.g., antibodies and soluble receptors) useful in the methods of the invention can be produced by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding, a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585.

In some embodiments a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular isolated polypeptide of interest will be inserted into an expression vector and operatively linked to an expression control sequence appropriate for expression of the protein in a desired host. Proper assembly can be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express NOTCH antagonist polypeptides (e.g., antibodies or soluble receptors). Recombinant expression vectors are replicable DNA constructs which have synthetic or cDNA-derived DNA fragments encoding a polypeptide of interest operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of a NOTCH antagonist polypeptide (e.g., antibody or soluble receptor) include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin as described below. Cell-free translation systems could also be employed. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference. Additional information regarding methods of protein production, including antibody production, can be found, e.g., in U.S. Patent Publication No 2008/0187954, U.S. Pat. Nos. 6,413,746 and 6,660,501, and International Patent Publication No WO 04009823, each of which is hereby incorporated by reference herein in its entirety.

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC, media, e.g., silica gel having pendant methyl or other aliphatic groups, can be employed to further purify a NOTCH antagonist polypeptide (e.g., antibody or soluble receptor). Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying a NOTCH antagonist polypeptide (e.g., antibody or soluble receptor) also include, for example, those described in U.S. Patent Publication No. 2008/0312425, 2008/0177048, and 2009/0187005, each of which is hereby incorporated by reference herein in its entirety.

### 6. Pharmaceutical Compositions

The NOTCH antagonist polypeptides (e.g., anti-NOTCH antibodies) can be formulated into a pharmaceutical composition by any suitable method known in the art. In certain embodiments, the pharmaceutical compositions comprise a pharmaceutically acceptable vehicle. The pharmaceutical compositions find use in inhibiting neuroendocrine tumor growth and treating neuroendocrine tumor in human patients.

In certain embodiments, formulations are prepared for storage and use by combining a purified NOTCH antagonist (e.g., an anti-NOTCH antibody) with a pharmaceutically acceptable vehicle (e.g. carrier, excipient) (Remington, The Science and Practice of Pharmacy 20th Edition Mack Publishing, 2000). Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

In certain embodiments, the pharmaceutical composition is frozen. In certain alternative embodiments, the pharmaceutical composition is lyophilized.

The pharmaceutical compositions of the present invention can be administered in any number of ways for either local or systemic treatment. Administration can be topical (such as to mucous membranes including vaginal and rectal delivery) such as transdermal patches, ointments, lotions, creams, gels, drops, suppositories, sprays, liquids and powders; pulmonary (e.g., by inhalation, or insufflation of powders or aerosols, including by nebulizer; intratracheal, intranasal, epidermal and transdermal); oral; or parenteral including intravenous, intraarterial, subcutaneous, intraperitoneal or intramuscular injection or infusion; or intracranial (e.g., intrathecal or intraventricular) administration.

The therapeutic formulation can be in unit dosage form. Such formulations include tablets, pills, capsules, powders, granules, solutions or suspensions in water or non-aqueous media, or suppositories for oral, parenteral, or rectal administration or for administration by inhalation. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and other diluents (e.g. water) to form a solid preformulation composition containing a homogeneous mixture of a compound of the present invention, or a non-toxic pharmaceutically acceptable salt thereof. The solid preformulation composition is then subdivided into unit dosage forms of the type described above. The tablets, pills, etc. of the novel composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials including a number of polymeric acids and mixtures of polymeric acids with such materials as shellac, cetyl alcohol and cellulose acetate.

The NOTCH antagonists (e.g., anti-NOTCH antibodies) can also be entrapped in microcapsules. Such microcapsules are prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions as described in Remington, The Science and Practice of Pharmacy 20th Ed. Mack Publishing (2000).

In certain embodiments, pharmaceutical formulations include the NOTCH antagonists (e.g., anti-NOTCH antibodies) complexed with liposomes (Epstein, et al., 1985, Proc. Natl. Acad. Sci. USA 82:3688; Hwang, et al., 1980, Proc. Natl. Acad. Sci. USA 77:4030; and U.S. Pat. Nos. 4,485,045 and 4,544,545). Liposomes with enhanced circulation time are disclosed in U.S. Pat. No. 5,013,556. Some liposomes can be generated by the reverse phase evaporation with a lipid composition comprising phosphatidylcholine, cholesterol, and PEG-derivatized phosphatidylethanolamine (PEG-PE). Liposomes are extruded through filters of defined pore size to yield liposomes with the desired diameter.

In addition sustained-release preparations can be prepared. Suitable examples of sustained-release preparations include semipermeable matrices of solid hydrophobic polymers containing the antibody, which matrices are in the form of shaped articles (e.g. films, or microcapsules). Examples of sustained-release matrices include polyesters, hydrogels such as poly(2-hydroxyethyl-methacrylate) or poly(vinylalcohol), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

### 7. Kits

Kits for practicing the methods of the invention are further provided. By “kit” is intended any manufacture (e.g., a package or a container) comprising at least one reagent, e.g., a nucleic acid probe, etc. for specifically detecting the level of NOTCH3 gene expression in a sample, e.g., cell, cell line, tumor, or tissue. The kit can be promoted, distributed, or sold as a unit for performing the methods of the present invention. Additionally, the kits can contain a package insert describing the kit and including instructional material for its use.

In one embodiment, kits for practicing the methods of the invention are provided. Such kits are compatible with both manual and automated screening. For qRT-PCR assays, the kits comprise at least the probes disclosed herein for the detection of NOTCH3 gene expression. The kits can further comprise reagents for RNA extraction, reverse transcription, and/or PCR amplifications. In certain embodiments, a kit according to the present invention comprises at least one oligonucleotide comprising a nucleotide sequence selected from the group consisting of SEQ ID NO:35-43.

Positive and/or negative controls can be included in the kits to validate the activity and correct usage of reagents employed in accordance with the invention. Controls can include samples, such as RNA preparations, formalin fixed tissues, etc., known to be either positive or negative for the presence of NOTCH3 mRNA. The design and use of controls is standard and well within the routine capabilities of those in the art.

It will be further appreciated that any or all steps in the methods of the invention could be implemented by personnel or, alternatively, performed in an automated fashion. Thus, the steps of body sample preparation, sample freezing or fixing, RNA extraction, and/or detection of NOTCH3 transcript level can be automated.

### EXAMPLES

It is understood that the examples and embodiments described herein are for illustrative purposes only and that various modifications or changes in light thereof will be suggested to persons skilled in the art and are to be included within the spirit and purview of this application.

### Example 1

### In Vivo Prevention of Tumor Growth Using the OMP-59R5 Anti-NOTCH2/3 Receptor Antibody as a Single Agent and in Combination with a Chemotherapeutic Agent

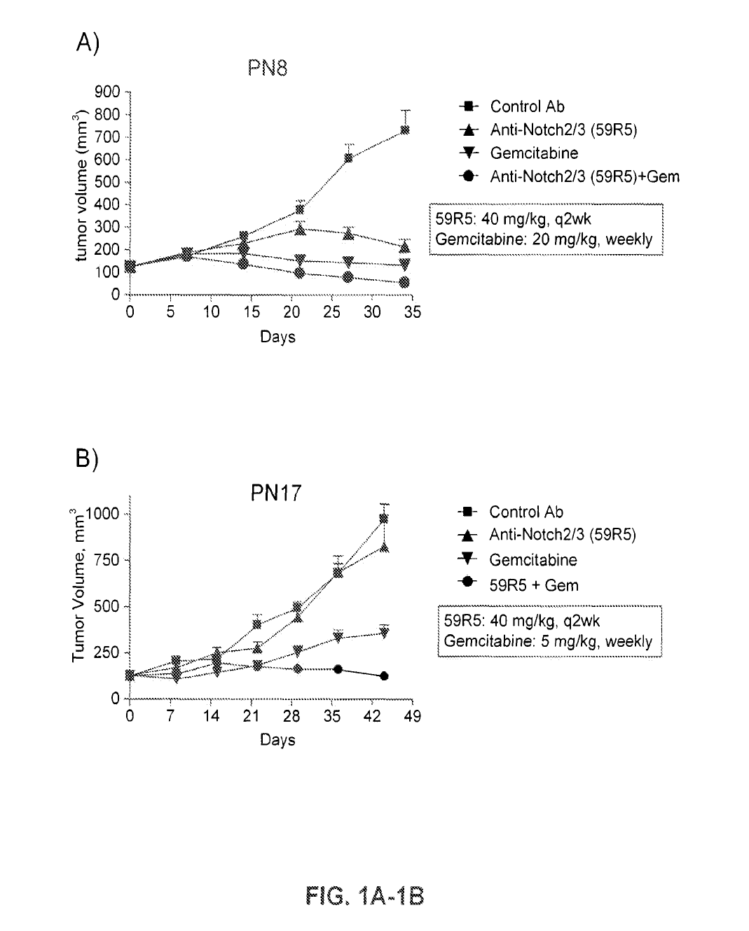

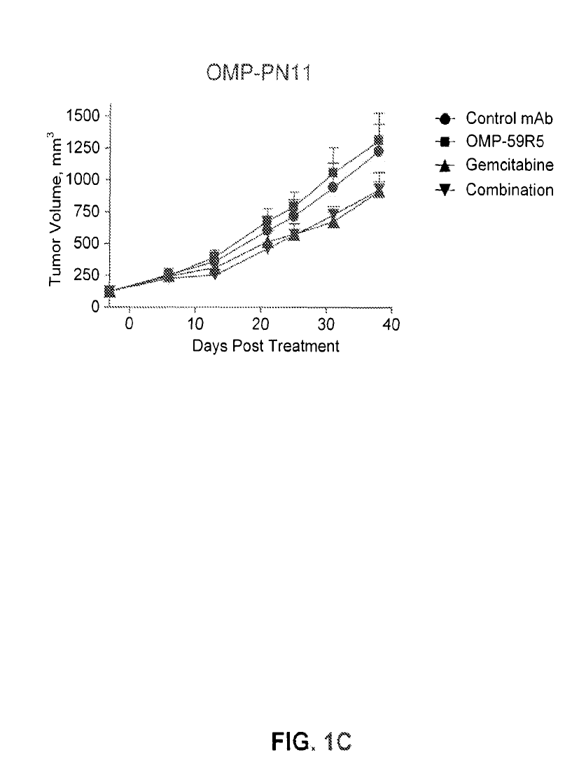

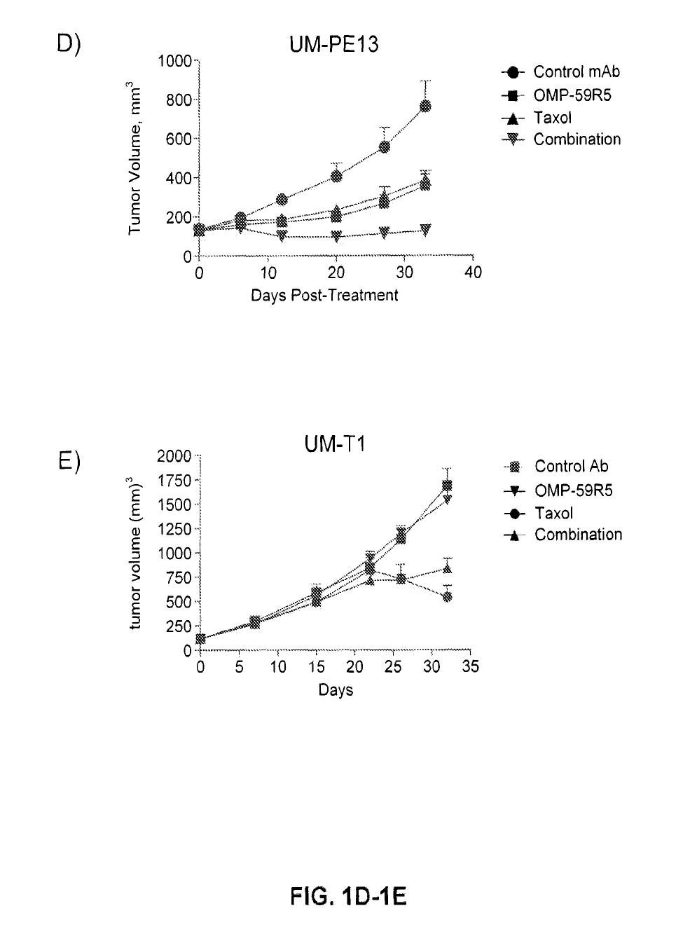

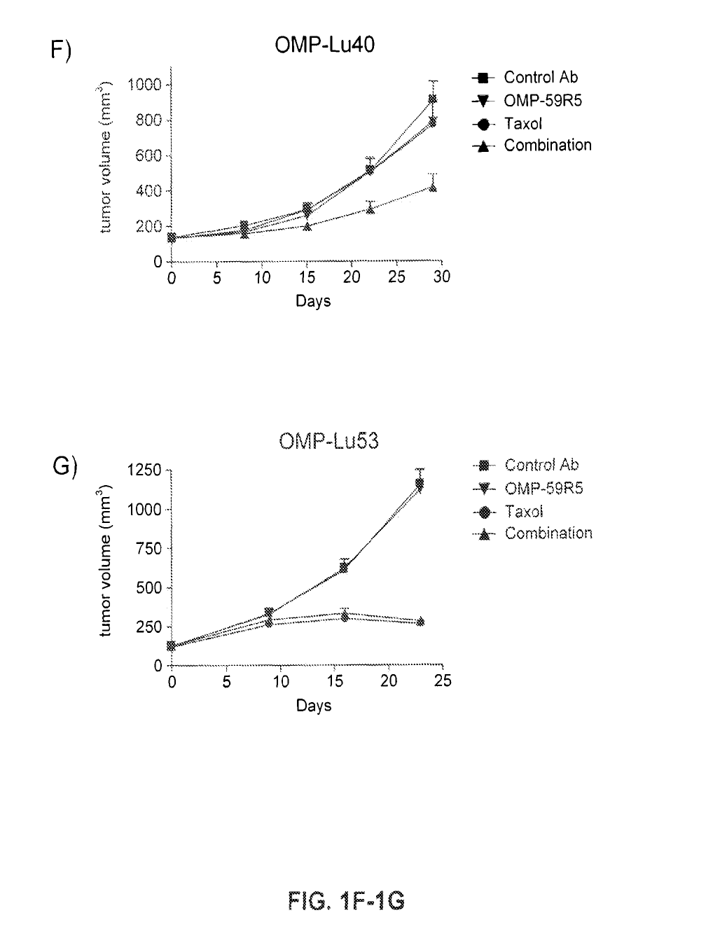

20,000 OMP-PN8 tumor cells were injected into NOD-SCID mice. Tumors were allowed to grow 22 days until they had reached an average volume of 125 mm³. Tumor bearing mice were randomized into 4 groups and treated with control antibody, OMP-59R5 (anti-NOTCH2/3), gemcitabine, or the combination of OMP-59R5 and gemcitabine. Antibodies were dosed every other week at 40 mg/kg. Gemcitabine was dosed at 20 mg/kg weekly. Tumor volumes were measured on the indicated days post-treatment. OMP-59R5 strongly inhibited OMP-PN8 tumor growth as a single agent or in combination with gemcitabine (FIG. 1A).

The ability of anti-NOTCH2/3 OMP-59R5 antibody to inhibit the in vivo growth of OMP-PN17 pancreatic tumor was determined using substantially identical methods. As shown in FIG. 1B, OMP-59R5 strongly inhibited OMP-PN17 tumor growth as a single agent or in combination with gemcitabine.

50,000 OMP-PN11 tumor cells were injected into NOD-SCID mice. Tumors were allowed to grow 21 days until they had reached an average volume of 120 mm³. Tumor bearing mice were randomized into 4 groups and treated with control antibody, OMP-59R5 (anti-NOTCH2/3), gemcitabine, or the combination of OMP-59R5 and gemcitabine. Antibodies were dosed every other week at 40 mg/kg. Gemcitabine was dosed at 20 mg/kg weekly. Tumor volumes were measured on the indicated days post-treatment. As shown in FIG. 1C, OMP-59R5 had no effect on OMP-PN11 tumor growth either as a single agent or in combination with gemcitabine.

20,000 UM-PE13 breast (NOTCH3 high expressing) tumor cells were injected into NOD-SCID mice. Tumors were allowed to grow 37 days until they had reached an average volume of 140 mm³. Tumor bearing mice were randomized into 4 groups and treated with control antibody, OMP-59R5, taxol, or the combination of OMP-59R5 and taxol. Antibodies were dosed weekly at 20 mg/kg. Taxol was dosed at 10 mg/kg weekly. Tumor volumes were measured on the indicated days post-treatment. As shown in FIG. 1D, OMP-59R5 strongly inhibited UM-PE 13 tumor growth as a single agent or in combination with taxol.

20,000 UM-T1 breast (NOTCH3 high expressing) tumor cells were injected into NOD-SCID mice. Tumors were allowed to grow 28 days until they had reached an average volume of 120 mm³. Tumor bearing mice were randomized into 4 groups and treated with either control antibody, OMP-59R5 anti-NOTCH2/3 antibody, taxol, or the combination of OMP-59R5 and taxol. Antibodies were dosed weekly at 20 mg/kg. Taxol was dosed at 10 mg/kg weekly. Tumor volumes were measured on the indicated days post-treatment. As shown in FIG. 1E, OMP-59R5 had no effect on UM-T1 tumor growth as a single agent or in combination with taxol.

50,000 OMP-Lu40 lung (NOTCH3 low expressing) tumor cells were injected into NOD-SCID mice. Tumors were allowed to grow 33 days until they had reached an average volume of 140 mm³. Tumor bearing mice were randomized into 4 groups and treated with either control antibody, OMP-59R5 anti-NOTCH2/3 antibody, taxol, or the combination of OMP-59R5 and taxol. Antibodies were dosed weekly at 20 mg/kg. Taxol was dosed at 10 mg/kg weekly. Tumor volumes were measured on the indicated days post-treatment. As shown in FIG. 1F, OMP-59R5 strongly inhibited OMP-Lu40 tumor growth in combination with taxol.

50,000 OMP-Lu53 lung (NOTCH3 high expressing) tumor cells were injected into NOD-SCID mice. Tumors were allowed to grow 33 days until they had reached an average volume of 120 mm³. Tumor bearing mice were randomized into 4 groups and treated with control antibody, OMP-59R5 anti-NOTCH2/3 antibody, taxol, or the combination of OMP-59R5 and taxol. Antibodies were dosed every other week at 40 mg/kg. Taxol was dosed at 10 mg/kg weekly. Tumor volumes were measured on the indicated days post-treatment. As shown in FIG. 1G, OMP-59R5 had no effect on OMP-Lu53 tumor growth in combination with taxol.

### Example 2

### Tumor Growth Inhibition by OMP-59R5 in Combination with Gemcitabine Significantly Correlates with the Levels of NOTCH3 Gene Expression in Pancreatic Tumors, but Not in Breast or Lung Tumors

NOTCH2 and NOTCH3 gene expression levels were determined in pancreatic, breast and lung tumors assayed in the in vivo xenograft assay described in Example 1 using standard microarray technology. Expression data was obtained using Affymetrix® U133 plus 2 arrays according to the manufacturer's instructions. The results are shown in Tables 1-3 below. The Tables also include data on the responsiveness of the particular tumor to treatment with OMP-59R5 anti-NOTCH2/3 antibody in combination with a chemotherapeutic agent in the in vivo xenograft assay described in Example 1. The analyses of NOTCH2 and 3 gene expression levels shown in the Tables were based on a cut-off value of 500. However, the overall conclusion from the analyses remained the same when the cut-off value was varied between 300 and 1000. No correlation between NOTCH3 expression and in vivo treatment efficacy was observed in the breast tumor and lung tumor samples: only 5 out of 14 breast or lung tumors with high NOTCH3 gene expression were responsive. Further, no correlation between NOTCH2 expression and in vivo efficacy was observed in breast, lung, or pancreatic tumor samples. Surprisingly, in pancreatic tumors there was a very strong correlation between high levels of NOTCH3 gene expression and the in vivo efficacy of OMP-59R5/gemcitabine treatment: 9 out of the 10 pancreatic tumors with high NOTCH3 gene expression were responsive in vivo to treatment with OMP-59R5 and gemcitabine.

TABLE 1
NOTCH2 and NOTCH3 gene expression levels in pancreatic tumors.

| | Efficacy | | |
|---|---|---|---|
| | (OMP-59R5 + | | |
| Tumor | gemcitabine) | N3 expression | N2 expression |
| PN4 | + | High (1802) | High (4637) |
| PN7 | − | Low (274) | High (2140) |
| PN8 | + | High (2484) | High (6909) |
| PN11 | − | Low (141) | High (4576) |
| PN13 | − | Low (23) | High (6848) |
| PN16 | + | High (3318) | High (3812) |
| PN17 | + | High (6106) | High (5904) |
| PN21 | + | High (2776) | High (6203) |
| PN23 | − | High (2978) | High (5166) |
| PN25 | + | High (6600) | High (4383) |

TABLE 2
NOTCH2 and NOTCH3 gene expression levels in breast tumors.

| | Efficacy | | |
|---|---|---|---|
| | (OMP-59R5 + | | |
| Tumor | taxol) | N3 expression | N2 expression |
| PE13 | + | High (5616) | High (6283) |
| T1 | − | High (11708) | High (7551) |
| B37 | + | High (10217) | High (3231) |
| B40 | − | High (11615) | High (10999) |

TABLE 3
NOTCH2 and NOTCH3 gene expression levels in lung tumors.
NSCLC—non-small cell lung cancer; SCLC—small cell lung cancer.

| | | Efficacy | | |
|---|---|---|---|---|
| | | (OMP-59R5 + | | |
| | Tumor | taxol) | N3 expression | N2 expression |
| NSCLC | Lu15 | − | Low (440) | High (1995) |
| NSCLC | Lu24 | − | High (5430) | High (3105) |
| NSCLC | Lu25 | − | High (9768) | High (3225) |
| NSCLC | Lu53 | − | High (12294) | High (7828) |
| SCLC | Lu40 | + | Low (423) | High (1040) |
| SCLC | Lu61 | + | High 11732 | High (1500) |
| SCLC | Lu65 | + | Low (269) | High (514) |
| SCLC | Lu66 | + | Low (9) | Low (12) |
| SCLC | Lu67 | − | High (682) | High (2214) |
| SCLC | Lu68 | + | High (838) | High (3519) |

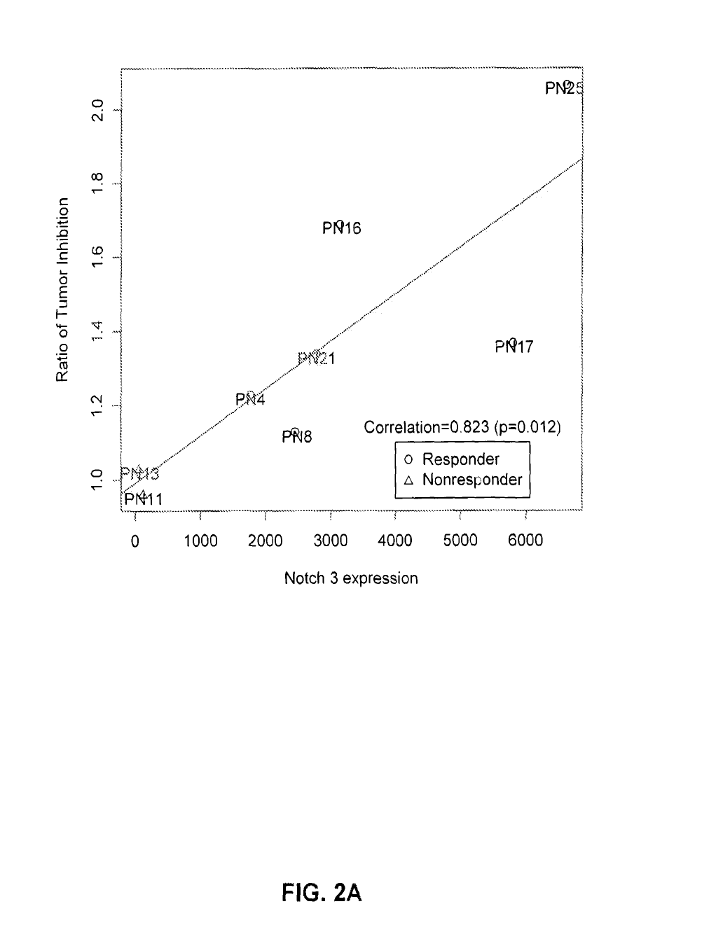

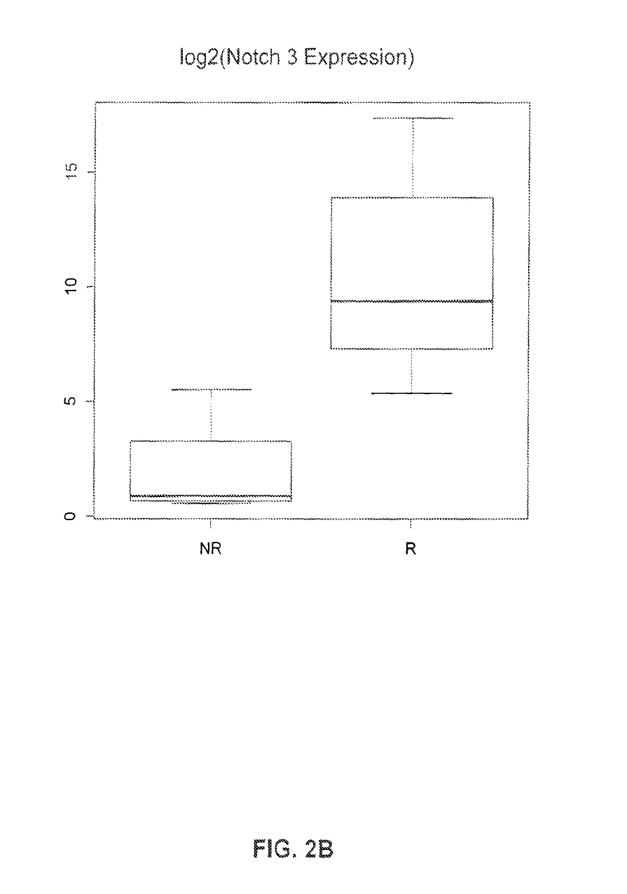

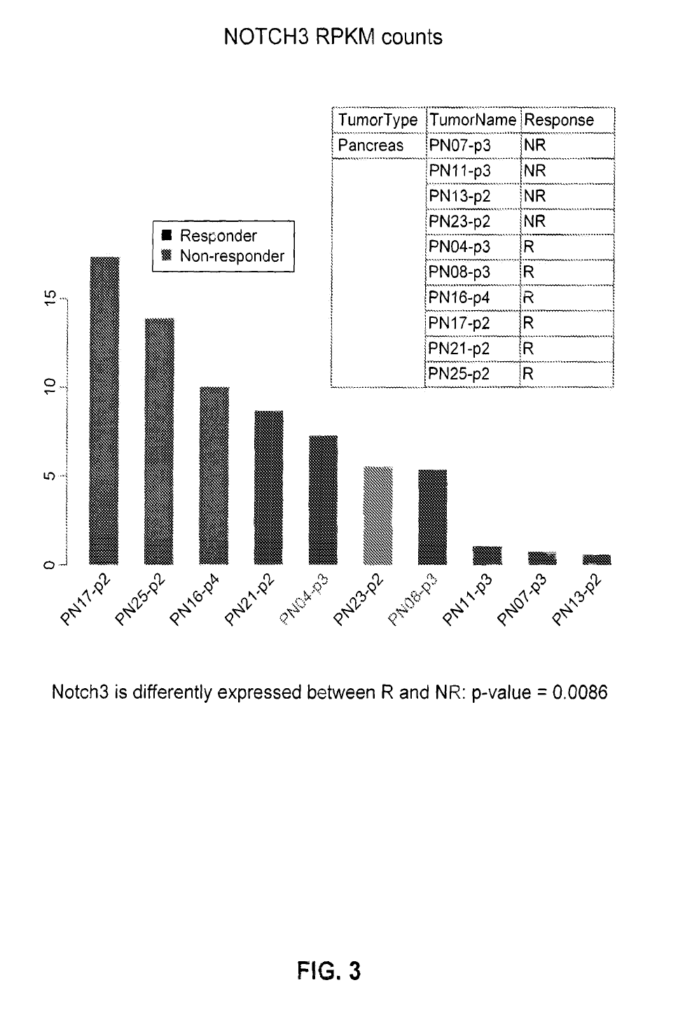

The surprising correlation between high levels of NOTCH3 gene expression and the in vivo efficacy of OMP-59R5/gemcitabine combination treatment in pancreatic tumors was further analyzed. NOTCH3 gene expression levels were determined in the PN11, PN13, PN23, PN04, PN08, PN16, PN17, PN21, and PN25 pancreatic tumor cells using standard multiplex transcript sequencing (e.g., RNASeq). RNASeq was performed using the Illumina® HiSeq™ 2000 Sequencing System according to the manufacturer's instructions. FIG. 2A shows that increased NOTCH3 gene expression significantly correlated (0.823; p<0.021) with in vivo tumor inhibition by OMP-59R5/gemcitabine combination treatment in human pancreatic xenograft models. FIG. 3 further shows that NOTCH3 gene expression detected in responsive pancreatic tumors was significantly higher than the expression level detected in non-responsive pancreatic tumors.

FIG. 2B shows the distribution of NOTCH3 gene expression detected in human pancreatic tumors which were responsive to treatment with OMP-59R5 anti-NOTCH2/3 antibody in combination with gemcitabine (R=responders: pval<0.05 compared to gemcitabine treatment alone) and for those xenografts which were found to be non-responsive to treatment with OMP-59R5 anti-NOTCH2/3 antibody in combination with gemcitabine (NR=non-responders: pval>0.05 compared to gemcitabine treatment alone). The distribution of NOTCH3 gene expression levels in non-responsive pancreatic tumors showed a clear separation from the distribution of NOTCH3 gene expression levels in responsive pancreatic tumors.

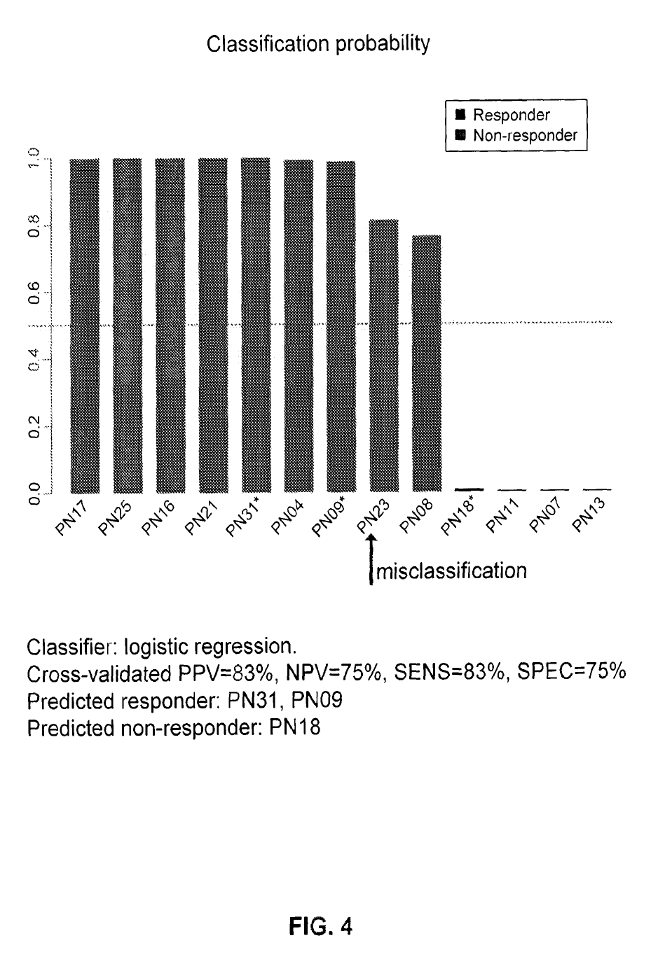

Logistic regression, a standard statistical model was used to predict the in vivo responsiveness of particular pancreatic cancers to treatment with OMP-59R5 in combination with a chemotherapeutic agent, e.g., gemcitabine, based on the NOTCH3 gene expression level detected in the pancreatic cancer by RNASeq. Alan Agresti: An Introduction to Categorical Data Analysis, John Wiley and Sons, Inc. (1996). Results of the analysis are shown in FIG. 4. The positive predictive value (PPV), negative predictive value (NPV), sensitivity (SENS) and specificity (SPEC) of the NOTCH3 gene expression data set was 83%, 75%, 83%, and 75%, respectively.

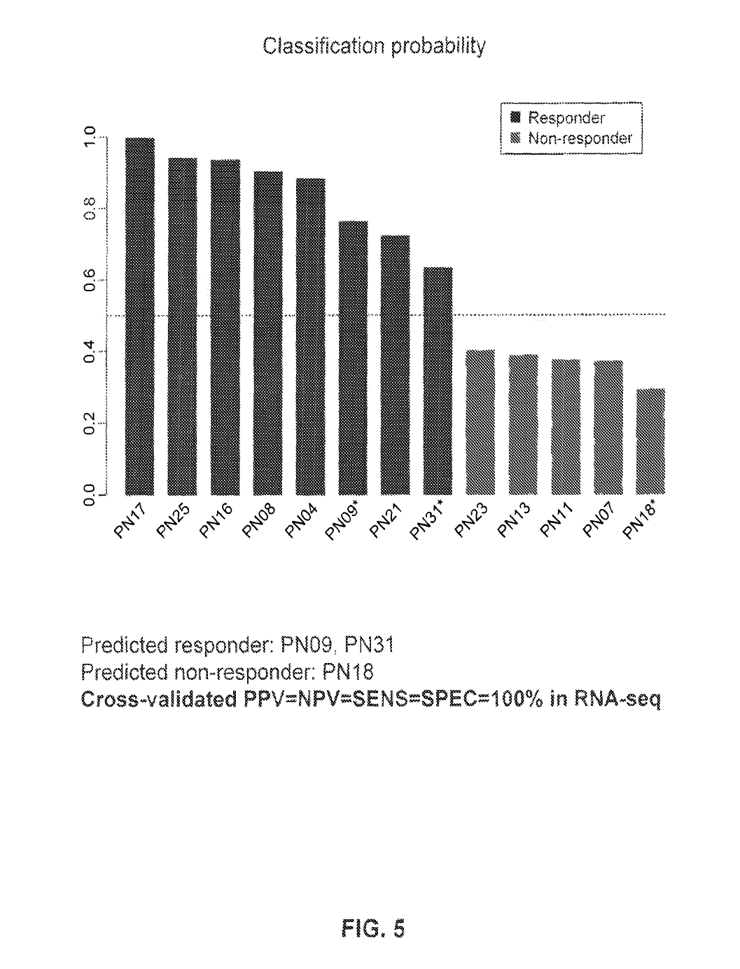

The accuracy of the prediction of in vivo responsiveness of pancreatic cancers to treatment with OMP-59R5 in combination with gemcitabine was further improved by including in the statistical analysis MAML2 gene expression data from the pancreatic cancers. The results obtained by applying logistic regression to the NOTCH3 and MAML2 gene expression data set are shown in FIG. 5. The positive predictive value (PPV), negative predictive value (NPV), sensitivity (SENS) and specificity (SPEC) of the NOTCH3 and MAML2 gene expression data set was 100%. The experiment was cross-validated using gene expression data obtained by standard RNASeq methods.

### Example 3

### NOTCH3 Protein Expression in Pancreatic Tumor Samples

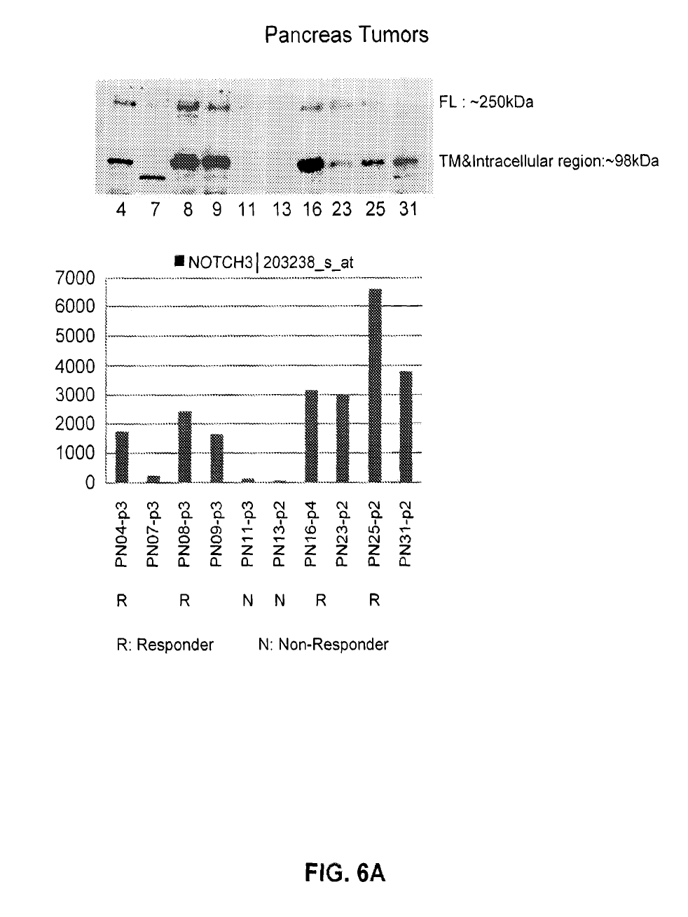

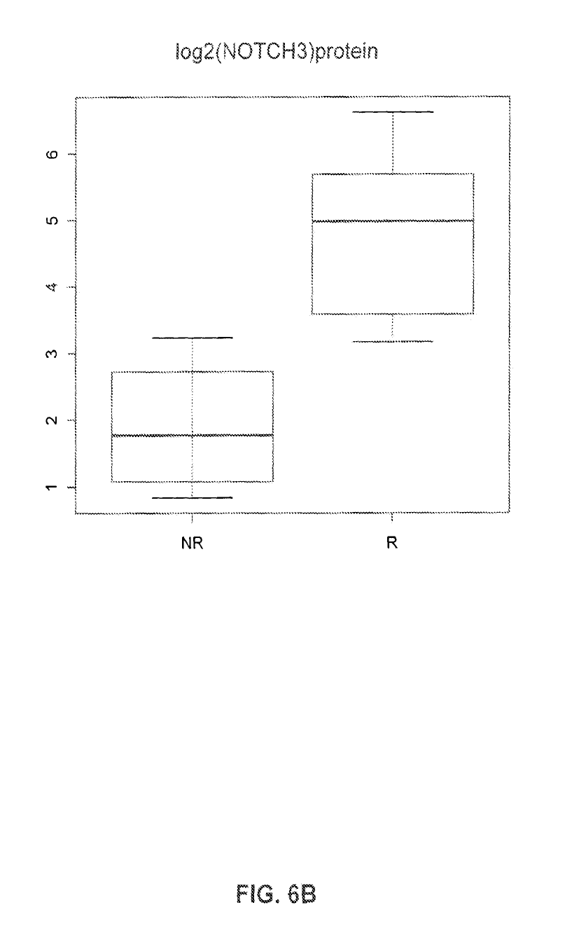

NOTCH3 Western blot analysis was performed to determine the expression of NOTCH3 protein in human pancreatic tumors (FIG. 6A). The anti-NOTCH3 antibody (Cell signaling #5276) used in this analysis detected both fall length NOTCH3 (FL: ˜250 kDa), and the transmembrane and intracellular regions of NOTCH3 (TM=˜98 kDa).

FIG. 6B shows the distribution of NOTCH3 protein expression in human pancreatic tumors which were responsive to treatment with OMP-59R5 in combination with gemcitabine (R=responders: pval<0.05 compared to gemcitabine treatment alone) and for those xenografts which were found to be non-responsive to treatment with OMP-59R5 in combination with gemcitabine (NR=non-responders: pval>0.05 compared to Gemcitabine treatment alone) in the xenograft assay described in Example 1. The separation in the distribution of NOTCH3 protein expression between responders and non-responders was less pronounced than the separation in the distribution of NOTCH3 gene expression. Logistic regression was applied to the NOTCH3 protein expression data in pancreatic cancers to predict the sensitivity of particular pancreatic cancers to treatment with OMP-59R5 in combination with gemcitabine. The NOTCH3 protein expression data generated similar performance in predicting the response to OMP-59R5 plus gemcitabine treatment to the performance of the NOTCH3 gene expression data discussed above.

### Example 4

### NOTCH3 Gene Expression in Metastatic Pancreatic Tumor Samples Measured by qRT-PCR

NOTCH3 gene expression was determined in metastatic pancreatic tumor samples using standard quantitative qRT-PCR. The assay probes were designed using the NOTCH3 RefSeq mRNA sequence NM_{—}000435.2. NOTCH3_A7 detects one of the two potential transcripts while NOTCH3_A1 detects both transcripts predicted by the Ensembl database. The probes and qRT-PCR assay were verified using human fresh frozen (FF) and formalin-fixed paraffin-embedded (FFPE) human tissue samples.

TABLE 3
Nucleotide sequence of probes used
in NOTCH3 qRT-PCR assays.

| | | |
|---|---|---|
| NOTHC3_A1 | Forward | AGGCAGAGTGGCGACCTC |
| | | (SEQ ID NO: 35) |
| | Reverse | CGTCCACGTTCACTTCACAATTC |
| | | (SEQ ID NO: 36) |
| | Probe | AACCCAGGAAGACAGGCACAGTCGT |
| | | (SEQ ID NO: 37) |
| NOTHC3_A9 | Forward | CTGGGTTTGAGGGTCAGAAT |
| | | (SEQ ID NO: 38) |
| | Reverse | GGGCACTGGCAGTTATAGGT |
| | | (SEQ ID NO: 39) |
| | Probe | TGACGCCATCCACGCATGTC |
| | | (SEQ ID NO: 40) |
| NOTCH3_A7 | Forward | TGCAGGATAGCAAGGAGGAGAC |
| | | (SEQ ID NO: 41) |
| | Reverse | GCAGCTTGGCAGCCTCATAG |
| | | (SEQ ID NO: 42) |
| | Probe | CTCGCGGGCGGCCAGGAATAGGG |
| | | (SEQ ID NO: 43) |

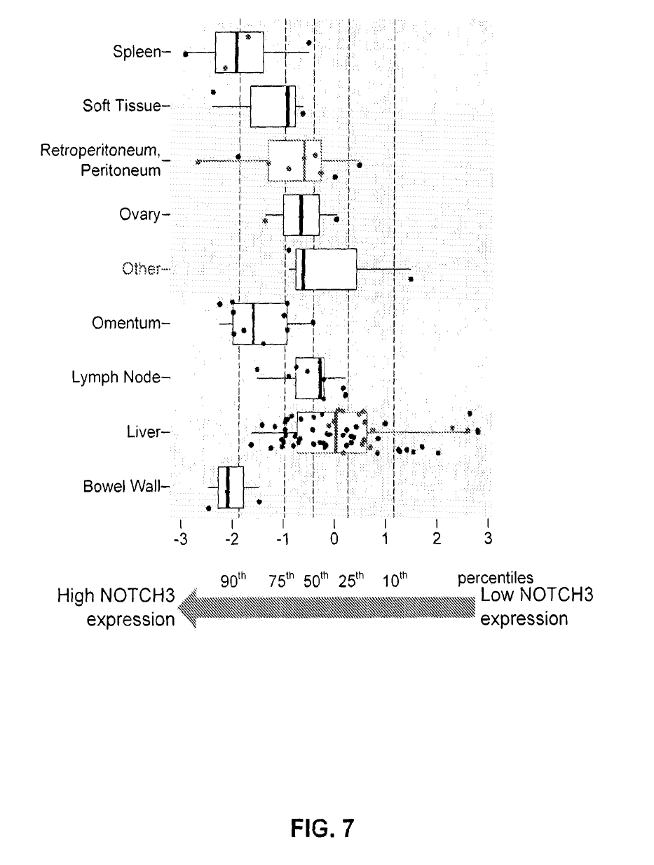

Approximately 100 formalin-fixed paraffin embedded (FFPE) metastatic tumor tissues from first-line pancreatic cancer patients were sourced to determine the levels and distribution of NOTCH3 expression in this cohort (FIG. 7). NOTCH3 gene expression was determined with the NOTCH3_A7 primer/probe set using a standard quantitative RT-PCR protocol. ANOVA statistical analysis was performed to determine if the levels of NOTCH3 correlated with factors including sample age, sex, patient age etc. NOTCH3 levels were not found to be correlated with any of these factors except for site of metastasis with liver showing significance and a wider NOTCH3 gene expression distribution. FIG. 7 displays the 10^{th}, 25^{th}, 50^{th}, 75^{th}, and 90^{th} percentile for NOTCH3 gene expression across all metastatic tumor samples examined.

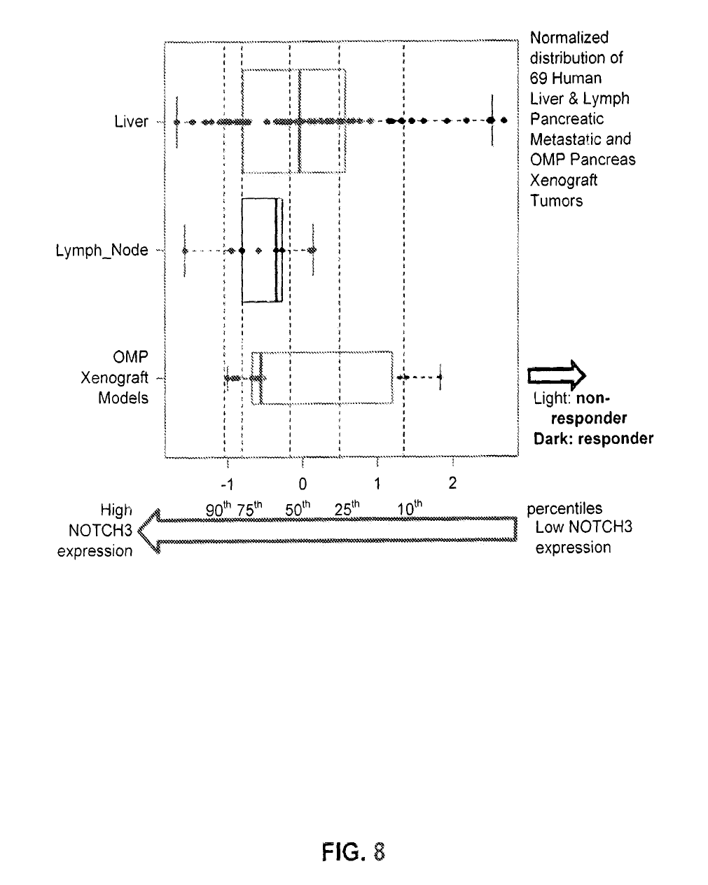

NOTCH3 gene expression levels from the sourced human liver and lymph node metastatic pancreatic cancer tissues and the primary human pancreatic tumors used in the xenograft assays were normalized in order to compare the data. The mean of data was subtracted and divided by the standard deviation in each data set. The grey (Light) dots represent the human pancreatic tumors that were non-responsive to treatment with OMP-59R5 in combination with gemcitabine in the xenograft assay described in Example 1, and the black (Dark) dots represent the human pancreatic tumors that were responsive in the xenograft assay (FIG. 8). The responsive tumors showed higher levels of NOTCH3 gene expression than the non-responsive ones, indicating that NOTCH3 gene expression can be used to predict in vivo responsiveness of pancreatic tumors to treatment with, for example, OMP-59R5 in combination with a chemotherapeutic agent. FIG. 8 also displays the 10^{th}, 25^{th}, 50^{th}, 75^{th}, and 90^{th} percentile for NOTCH3 gene expression in the human liver and lymph node metastatic pancreatic cancer tissues examined.

### Example 6

### The OMP-59R5 Anti-NOTCH2/3 Antibody in Combination with Gemcitabine and ABRAXANE™ Inhibits in Vitro Growth of Pancreatic Tumors

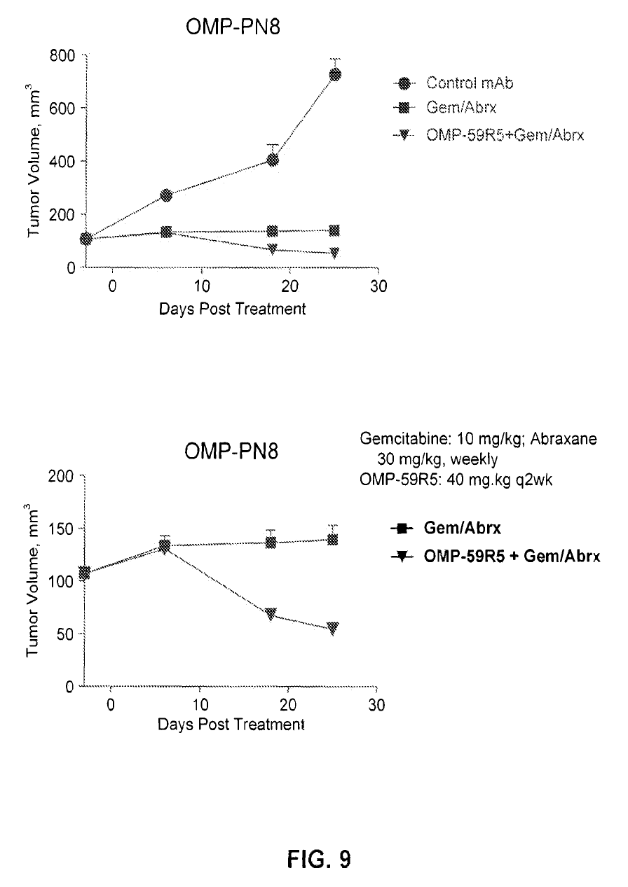

20,000 OMP-PN8 (NOTCH3 high expressing) tumor cells were injected into NOD-SCID mice. Tumors were allowed to grow 26 days until they had reached an average volume of 110 mm³. Tumor bearing mice were randomized into 3 groups (n=9 mice per group) and treated with control antibody, gemcitabine, plus ABRAXANE™ (albumin bound paclitaxel), or the combination of OMP-59R5 anti-NOTCH2/3 antibody and gemcitabine plus ABRAXANE™ OMP-59R5 was dosed every other week at 40 mg/kg. Gemcitabine was dosed at 10 mg/kg weekly and ABRAXANE™ at 30 mg/kg weekly. Tumor volumes were measured on the indicated days post-treatment. OMP-59R5 strongly inhibited OMP-PN8 tumor growth in combination with gemcitabine plus ABRAXANE™, and was more active than gemcitabine plus ABRAXANE™ alone (FIG. 9). The top and bottom graphs show data obtained from the same experiment on different scales. The bottom graph shows data obtained from the active treatment groups only, but not data obtained from control treated animals. The results indicate that NOTCH3 expression levels can be used to predict in vivo responsiveness of pancreatic tumors to treatment with OMP-59R5 antibody in combination with various chemotherapeutic agents.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

SEQUENCES

SEQ ID NO: 1
HKGAL
SEQ ID NO: 1
HEDAI
SEQ ID NO: 3: 59R1Heavy chain CDR1
SSSGMS
SEQ ID NO: 4: 59R1Heavy chain CDR2
VIASSGSNTYYADSVKG
SEQ ID NO: 5: 59R1Heavy chain CDR3
GIFFAI
SEQ ID NO: 6: 59R1 Light chain CDR1
RASQSVRSNYLA
SEQ ID NO: 7: 59R1 Light chain CDR2
GASSRAT
SEQ ID NO: 8: 59R1 Light chain CDR3
QQYSNFPI
SEQ ID NO: 9: 59R5 Heavy chain CDR3
SIFYTT
SEQ ID NO: 10 (heavy chain CDR3 consensus sequence):
(G/S) (I/S)F(F/Y) (A/P) (I/T/S/N)
SEQ ID NO: 11 (alternative heavy chain CDR3)
SIFYPT
SEQ ID NO: 12 (alternative heavy chain CDR3)
SSFFAS
SEQ ID NO: 13 (alternative heavy chain CDR3)
SSFYAS
SEQ ID NO: 14 (alternative heavy chain CDR3)
SSFEAT
SEQ ID NO: 15 (alternative heavy chain CDR3)
SIFYPS
SEQ ID NO: 16 (alternative heavy chain CDR3)
SSFFAN
SEQ ID NO: 17: 59R5 Heavy chain variable region
EVQLVESGGGLVQPGGSLRLSCAASGFTFSSSGMSWVRQAPGKGLEWVSVIASSGSNTYYADSVKGRF
TISRDNSKNTLYLQMNSLRAEDTAVYYCARSIFYTTWGQGTLVTVSSAST
SEQ ID NO: 18: 59R1 Heavy chain VH of 59R1 IgGantibody
QVQLVESGGGLVQPGGSLRLSCAASGFTFSSSGMSWVRQAPGKGLEWVSVIASSGSNTYYADSVKGRF
TISRDNSKNTLYLQMNSLRAEDTAVYYCARGIFFAIWGQGTLVTVSSA
SEQ ID NO: 19: 59R1 heavy chain VH plus mammalian signal sequence
(underlined)
MKHLWFFLLLVAAPRWVLSQVQLVESGGGLVQPGGSLRLSCAASGFTFSSSGMSWVRQAPGKGLEWVS
VIASSGSNTYYADSVKGRFTISRDNSKNTLYLQMNSLRAEDTAVYYCARGIFFAIWGQGTLVTVSSA
SEQ ID NO: 20: Variant 59R1 Heavy chain variable region
QVQLVESGGGLVQPGGSLRLSCAASGFTFSSSGMSWVRQAPGKGLEWVSVIASSGSNTYYADSVKGRF
TISRDNSKNTLYLQMNSLRAEDTAVYYCARSIFYPTWGQGTLVTVSSA
SEQ ID NO: 21: Variant 59R1 Heavy chain variable region
QVQLVESGGGLVQPGGSLRLSCAASGETFSSSGMSWVRQAPGKGLEWVSVIASSGSNTYYADSVKGRF
TISRDNSKNTLYLQMNSLRAEDTAVYYCARSSFFASWGQGTLVTVSSA
SEQ ID NO: 22: Variant 59R1 Heavy chain variable region
QVQLVESGGGLVQPGGSLRLSCAASGFTFSSSGMSWVRQAPGKGLEWVSVIASSGSNTYYADSVKGRF
TISRDNSKNTLYLQMNSLRAEDTAVYYCARSSFYASWGQGTLVTVSSA
SEQ ID NO: 23: Variant 59R1 Heavy chain variable region
QVQLVESGGGLVQPGGSLRLSCAASGFTFSSSGMSWVRQAPGKGLEWVSVIASSGSNTYYADSVKGRF
TISRDNSKNTLYLQMNSLRAEDTAVYYCARSSFFATWGQGTLVTVSSA
SEQ ID NO: 24: Variant 59R1 Heavy chain variable region
QVQLVESGGGLVQPGGSLRLSCAASGFTFSSSGMSWVRQAPGKGLEWVSVIASSGSNTYYADSVKGRF
TISRDNSKNTLYLQMNSLRAEDTAVYYCARSIFYPSWGQGTLVTVSSA
SEQ ID NO: 25: Variant 59R1 Heavy chain variable region
QVQLVESGGGLVQPGGSLRLSCAASGFTFSSSGMSWVRQAPGKGLEWVSVIASSGSNTYYADSVKGRF
TISRDNSKNTLYLQMNSLRAEDTAVYYCARSSFFANWGQGTLVTVSSA
SEQ ID NO: 26: 59R1 Heavy chain VH of 59RGV antibody (germlined
variant of 59R1)
EVQLVESGGGLVQPGGSLRLSCAASGFTFSSSGMSWVRQAPGKGLEWVSVIASSGSNTYYADSVKGRF
TISRDNSKNTLYLQMNSLRAEDTAVYYCARGIFFAIWGQGTLVTVSSA
SEQ ID NO: 27: 59R1 Light chain VL of 59RGV antibody (germlined
variant of 59R1)
EIVLTQSPATLSLSPGERATLSCRRASQSVRSNYLAWYQQKPGQAPRLLIYGASSRATGIPARFSGSG
SGTDFTLTISSLEPEDFAVYYCQQYSNFPITFGQGTKVEIKR
SEQ ID NO: 28: 59R1 light chain VL plus mammalian signal, sequence
(underlined)
MVLQTQVFISLLLWISGAYGDIVLTQSPATLSLSPGERATLSCRASQSVRSNYLAWYQQKPGQAPRLL
IYGASSRATGVPARFSGSGSGTDFTLTISSLEPEDFAVYYCQQYSNFPITFGQGTKVEIKR
SEQ ID NO: 29: 59R1 Light chain VL of 59R1 IgG antibody
DIVLTQSPATLSLSPGERATLSCRASQSVRSNYLAWYQQKPGQAPRLLIYGASSRATGVPARFSGSGS
GTDFTLTISSLEPEDFAVYYCQQYSNFPITFGQGTKVEIKR
SEQ ID NO: 30: 59R5 Heavy chain
EVQLVESGGGLVQPGGSLRLSCAASGFTFSSSGMSWVRQAPGKGLEWVSVIASSGSNTYYADSVKGRF
TISRDNSKNTLYLQMNSLRAEDTAVYYCARSIFYTTWGQGTLVTVSSASTKGPSVFPLAPCSRSTSES
TAALGCLVKDYFPEPVTVSWNSGALTSGVHTFPAVLQSSGLYSLSSVVTVPSSNFGTQTYTCNVDHKP
SNTKVDKTVERKCCVECPPCPAPPVAGPSVFLFPPKPKDTLMISRTPEVTCVVVDVSHEDPEVQFNWY
VDGVEVHNAKTKPREEQFNSTFRVVSVLTVVHQDWLNGKEYKCKVSNKGLPAPIEKTISKTKGQPREP
QVYTLPPSREEMTKNQVSLTCLVKGFYPSDIAVEWESNGQPENNYKTTPPMLDSDGSFFLYSKLTVDK
SRWQQGNVFSCSVMHEALHNHYTQKSLSLSPGK
SEQ ID NO: 31: Predicted protein sequence of anti-NOTCH2/3 59R1 IgG2
heavy chain, plus signal sequence. The signal sequence is underlined.
MKHLWFFLLLVAAPRWVLSQVQLVESGGGLVQPGGSLRLSCAASGFTFSSSGMSWVRQAPGKGLEWVS
VIASSGSNTYYADSVKGRFTISRDNSKNTLYLQMNSLRAEDTAVYYCARGIFFAIWGQGTLVTVSSAS
TKGPSVFPLAPCSRSTSESTAALGCLVKDYFPEPVTVSWNSGALTSGVHTFPAVLQSSGLYSLSSVVT
VPSSNFGTQTYTCNVDHKPSNTKVDKTVERKCCVECPPCPAPPVAGPSVFLFPPKPKDTLMISRTPEV
TCVVVDVSHEDPEVQFNWYVDGVEVHNAKTKPREEQFNSTFRVVSVLTVVHQDWLNGKEYKCKVSNKG
LPAPIEKTISKTKGQPREPQVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLSPGK
SEQ ID NO: 32: Predicted protein sequence of the heavy chain of
anti-NOTCH2/3 59RGV (germlined variant of 59R1), plus signal
sequence. The signal sequence is underlined.
MKHLWFFLLLVAAPRWVLSEVQLVESGGGLVQPGGSLRLSCAASGFTFSSSGMSWVRQAPGKGLEWVS
VIASSGSNTYYADSVKGRFTISRDNSKNTLYLQMNSLRAEDTAVYYCARGIFFAIWGQGTLVTVSSAS
TKGPSVFPLAPCSRSTSESTAALGCLVKDYFPEPVTVSWNSGALTSGVHTFPAVLQSSGLYSLSSVVT
VPSSNFGTQTYTCNVDHKPSKTKVDKTVERKCCVECPPCPAPPVAGPSVFLFPPKPKDTLMISRTPEV
TCVVVDVSHEDPEVQFNWYVDGVEVHNAKTKPREEQFNSTFRVVSVLTVVHQDWLNGKEYKCKVSNKG
LPAPIEKTISKTKGQPREPQVYTLPPSREEMTKNQVSLTCLVKGFYPSDIAVEWESNGQPENNYKTTP
PML'DSDGSFFLYSKLTVDKSRWQQGNVFSCSVMHEALHNHYTQKSLSLSPGK
SEQ ID NO: 33: Predicted protein sequence of anti-NOTCH2/3 59R1 light
chain, plus signal sequence. The signal sequence is underlined.
MVLQTQVFISLLLWISGAYGDIVLTQSPATLSLSPGERATLSCRASQSVRSNYLAWYQQKPGQAPRLL
IYGASSRATGVPARFSGSGSGTDFTLTISSLEPEDFAVYYCQQYSNFPITFGQGTKVEIKRTVAAPSV
FIFPPSDEQLKSGTASVVCLLNNFYPREAKVQWKVDNALQSGNSQESVTEQDSKDSTYSLSSTLTLSK
ADYEKHKVYACEVTHQGLSSPVTKSFNRGEC
SEQ ID NO:34: Predicted protein sequence of the light chain of
anti-NOTCH2/3 59RGV antibody (germlined variant of 59R1), plus
signal sequence. The signal sequence is underlined.
MVLQTQVFISLLLWISGAYGEIVLTQSPATLSLSPGERATLSCRRASQSVRSNYLAWYQQKPGQAPRL
LIYGASSRATGIPARFSGSGSGTDFTLTISSLEPEDFAVYYCQQYSNFPITFGQGTKVEIKRTVAAPS
VFIFPPSDEQLKSGTASVVOLLNNFYPREAKVQWKVDNALQSGNSQESVTEQDSKDSTYSLSSTLTLS
KADYEKHKVYACEVTHQGLSSPVTKSFNRGEC
SEQ ID NO: 35
AGGCAGAGTGGCGACCTC
SEQ ID NO: 36
CGTCCACGTTCACTTCACAATTC
SEQ ID NO: 37
AACCCAGGAAGACAGGCACAGTCGT
SEQ ID NO: 38
CTGGGTTTGAGGGTCAGAAT
SEQ ID NO: 39
GGGCACTGGCAGTTATAGGT
SEQ ID NO: 40
TGACGCCATCCACGCATGTC
SEQ ID NO: 41
TGCAGGATAGCAAGGAGGAGAC
SEQ ID NO: 42
GCAGCTTGGCAGCCTCATAG
SEQ ID NO: 43
CTCGCGGGCGGCCAGGAATAGGG

What we claim is:

1. A method for selecting a pancreatic cancer patient for treatment with a NOTCH inhibitor comprising: (a) determining the level of expression of one or more biomarkers in tumor cells from said patient, wherein the one or more biomarkers comprise NOTCH3, and (b) selecting the patient based on the expression level of the one or more biomarkers.

2. A method for determining whether a patient diagnosed with pancreatic cancer is likely to respond to a NOTCH inhibitor-based therapy comprising determining the level of expression of one or more biomarkers in tumor cells from said patient, wherein the one or more biomarkers comprise NOTCH3, and the level of expression of the one or more biomarkers indicates that the patient is likely to respond to therapy.

3. A method for determining whether a patient diagnosed with pancreatic cancer should be administered a NOTCH inhibitor, comprising determining the level of expression of one or more biomarkers in tumor cells from said patient, wherein the one or more biomarkers comprise NOTCH3, and the level of expression of the one or more biomarkers is predictive of said patient having a favorable response to treatment with a NOTCH inhibitor.

4. A method to determine whether a patient diagnosed with pancreatic cancer should continue treatment with a NOTCH inhibitor, comprising determining the level of expression of one or more biomarkers in tumor cells from said patient, wherein the one or more biomarkers comprise NOTCH3, and the level of expression of the one or more biomarkers indicates that the patient is likely to respond to therapy.

5. A method to determine whether a patient diagnosed with pancreatic cancer should continue treatment with a NOTCH inhibitor, comprising determining the level of expression of one or more biomarkers in tumor cells from said patient, wherein the one or more biomarkers comprise NOTCH3, and the level of expression of the one or more biomarkers is predictive of said patient having a favorable response to treatment with said NOTCH inhibitor.

6. A method for determining the therapeutic efficacy of a NOTCH inhibitor for treating pancreatic cancer in a patient comprising determining the level of expression of one or more biomarkers in tumor cells from said patient, wherein the one or more biomarkers comprise NOTCH3, and the level of expression of the one or more biomarkers is indicative of the therapeutic efficacy of said NOTCH inhibitor.

7. A method of treating pancreatic cancer in a patient comprising:
(a) determining the level of expression of one or more biomarkers in tumor cells from said patient, wherein the one or more biomarkers comprise NOTCH3; and
(b) administering to said patient a therapeutically effective amount of a NOTCH inhibitor.

8. A method for stratifying a pancreatic cancer patient population for treatment with a NOTCH inhibitor comprising:
(a) determining the level of expression of one or more biomarkers in tumor cells from said patients, wherein the one or more biomarkers comprise NOTCH3, and
(b) stratifying the patient population based on the level of expression of the one or more biomarkers in the tumor cells.

9. The method of any one of claims 1-8, wherein the level of NOTCH3 expression is determined to be above a reference level for NOTCH3 expression.

10. The method of any one of claims 1-9, wherein each of the biomarkers is determined to be expressed at a level above a reference level for the biomarker.

11. The method of any one of claims 1-10, wherein the expression level of the one or more biomarkers is determined by determining the level of the biomarker mRNA or the biomarker protein.

12. The method of any one of claims 1-11, wherein the level of NOTCH3 expression is determined by determining the level of NOTCH3 mRNA in the tumor cells.

13. The method of claim 12, wherein the NOTCH3 mRNA level is determined by quantitative polymerase chain reaction.

14. The method of claim 13, wherein the NOTCH3 mRNA level is determined using: (a) a forward primer having a nucleotide sequence selected from the group consisting of SEQ ID NO: 35, SEQ ID NO: 38, and SEQ ID NO: 41; (b) a reverse primer having a nucleotide sequence selected from the group consisting of SEQ ID NO: 36, SEQ ID NO: 39, and SEQ ID NO: 42; and/or (c) a probe comprising an oligonucleotide having a nucleotide sequence selected from the group consisting of SEQ ID NO: 37, SEQ ID NO: 40, and SEQ ID NO: 43.

15. The method of claim 14, wherein the NOTCH3 mRNA level is determined using: (a) a forward primer having the sequence of SEQ ID NO: 35, a reverse primer having the sequence of SEQ ID NO: 36, and a probe comprising an oligonucleotide having the sequence of SEQ ID NO: 37;
(b) a forward primer having the sequence of SEQ ID NO: 38, a reverse primer having the sequence of SEQ ID NO: 39, and a probe comprising an oligonucleotide having the sequence of SEQ ID NO: 40; or
(c) a forward primer having the sequence of SEQ ID NO: 41, a reverse primer having the sequence of SEQ ID NO: 42, and a probe comprising an oligonucleotide having the sequence of SEQ ID NO: 43.

16. The method of claim 12, wherein the NOTCH3 mRNA level is determined by array hybridization.

17. The method of any one of claims 1-11, wherein the level of NOTCH3 expression is determined by determining the level of NOTCH3 protein expressed by the tumor cells.

18. The method of any one of claims 1-17, wherein the one or more biomarkers consist of NOTCH3.

19. The method of any one of the claims 1-17, wherein the one or more biomarkers further comprise MAML2 and the level of MAML2 expression is determined to be above a reference level for MAML2 expression.

20. The method of claim 19 wherein the one or more biomarkers consist of NOTCH3 and MAML2 expression.

21. The method of claim 19 or 20, wherein the level of MAML2 expression is determined by determining the level of MAML2 mRNA in the tumor cells.

22. The method of claim 19 or 20, wherein the level of MAML2 expression is determined by determining the level of MAML2 protein expressed by the tumor cells.

23. A method of treating pancreatic cancer in a patient comprising administering to said patient a therapeutically effective amount of a NOTCH inhibitor, wherein at least some of the pancreatic tumor cells from said patient express each of one or more biomarkers at a level above a reference level for that biomarker and/or have been previously determined to express each of one or more biomarkers at a level above a reference level for that biomarker, wherein the one or more biomarkers comprise NOTCH3.

24. The method of claim 23, wherein the level of NOTCH3 expression is determined as the level of NOTCH3 mRNA.

25. The method of claim 23, wherein the level of NOTCH3 expression is determined as the level of NOTCH3 protein.

26. The method of any one of claims 23-25, wherein the one or more biomarkers consist of NOTCH3.

27. The method of any one of the claims 23-25, wherein the one or more biomarkers further comprise MAML2 and the level of MAML2 expression is above a reference level for MAML2 expression.

28. The method of claim 27 wherein the one or more biomarkers consist of NOTCH3 and MAML2.

29. The method of any one of claims 1-28, wherein the reference level of a biomarker is a predetermined value.

30. The method of any one of claims 1-29, wherein the reference level of a biomarker is the level of expression of that biomarker in a control sample.

31. The method of any one of the claims 1-29, wherein the reference level for NOTCH3 expression is the 25^{th} percentile, the 30^{th} percentile, the 40^{th} percentile, the 50^{th} percentile, the 60^{th} percentile, the 70^{th} percentile, the 75^{th} percentile, or the 80^{th} percentile for NOTCH3 expression in pancreatic cancers or a subset of pancreatic cancers.

32. The method of any one of the claims 1-29, wherein the reference level for NOTCH3 expression is the 75^{th} percentile for NOTCH3 expression in pancreatic cancers.

33. The method of any one of the claims 1-29, wherein the reference level for NOTCH3 expression is the 50^{th} percentile for NOTCH3 expression in pancreatic cancers.

34. The method of any one of the claims 1-29, wherein the reference level for NOTCH3 expression is the 25^{th} percentile for NOTCH3 expression in pancreatic cancers.

35. The method of any one of claims 1-29, wherein the reference level for NOTCH3 expression is the 75^{th} percentile for NOTCH3 expression in pancreatic adenocarcinomas, metastatic pancreatic tumors, liver and/or lymph node metastatic pancreatic tumors, or chemotherapy-resistant pancreatic cancers.

36. The method of any one of claims 1-29, wherein the reference level for NOTCH3 expression is the 50^{th} percentile for NOTCH3 expression in pancreatic adenocarcinomas, metastatic pancreatic tumors, liver and/or lymph node metastatic pancreatic tumors or chemotherapy-resistant pancreatic cancers.

37. The method of any one of claims 1-29, wherein the reference level for NOTCH3 expression is the 25^{th} percentile for NOTCH3 expression in pancreatic adenocarcinomas, metastatic pancreatic tumors, liver and/or lymph node metastatic pancreatic tumors or chemotherapy-resistant pancreatic cancers.

38. The method of any of claim 1-22, or 29-37, further comprising obtaining a body sample from said patient.

39. The method of any of claims 1-38, wherein the level of expression of NOTCH3 is the level in a body sample from the patient.

40. The method of claim 38 or 39, wherein said sample is whole blood, plasma, serum, or tissue.

41. The method of claim 38, 39, or 40, wherein said sample is a pancreatic tumor sample.

42. The method of claim 41, wherein the sample is from a pancreatic tumor that has metastasized to the liver.

43. The method of any one of claims 38-42, wherein the sample is formalin-fixed paraffin embedded (FFPE) tissue.

44. The method of any of claims 1-43, wherein said patient is a human or said patient population is a human population.

45. The method of any of claims 1-44, wherein said pancreatic cancer is adenocarcinoma.

46. The methods of any one of claims 1-45, wherein the pancreatic cancer is chemotherapy-resistant.

47. The method of any of claim 1-6, 8-22, or 29-46, further comprising administering the NOTCH inhibitor to said patient.

48. The method of any of claims 1-47, wherein said NOTCH inhibitor is a gamma-secretase inhibitor.

49. The method of any of claims 1-47, wherein said NOTCH inhibitor is an anti-NOTCH antibody.

50. The method of claim 49, wherein said anti-NOTCH antibody is a monoclonal antibody.

51. The method of claim 49 or 50, wherein said anti-NOTCH antibody specifically binds to human NOTCH2 or human NOTCH3.

52. The method of claim 51, wherein said anti-NOTCH antibody specifically binds to human NOTCH2 and NOTCH3.

53. The method of claim 49 or 50, wherein said anti-NOTCH antibody specifically binds to EGF repeat 10 of human NOTCH2.

54. The method of claim 49 or 50, wherein said anti-NOTCH antibody specifically binds to EGF repeat 9 of human NOTCH3.

55. The method of any claim 52, wherein said anti-NOTCH antibody comprises an antigen-binding site that binds both the EGF repeat 9 of human NOTCH3 and the EGF repeat 10 of NOTCH2.

56. The method of any one of claims 1-55, wherein said NOTCH inhibitor is an antagonist of human NOTCH2 and/or NOTCH3.

57. The method of any one of claims 1-56, wherein said NOTCH inhibitor inhibits binding of a ligand to human NOTCH2 and/or NOTCH3.

58. The method of any one of claims 1-57, wherein said NOTCH inhibitor inhibits signaling of human NOTCH2 and/or NOTCH3.

59. The method of claim 52, wherein said anti-NOTCH antibody is encoded by the polynucleotide deposited with ATCC as PTA-9547.

60. The method of claim 49 or 50, wherein said anti-NOTCH antibody specifically binds human NOTCH2 and/or NOTCH3, wherein the antibody comprises:
(a) a heavy chain CDR1 comprising SSSGMS (SEQ ID NO:3), a heavy chain CDR2 comprising VIASSGSNTYYADSVKG (SEQ ID NO:4), and a heavy chain CDR3 comprising SIFYTT (SEQ ID NO:9); and
(b) a light chain CDR1 comprising RASQSVRSNYLA (SEQ ID NO:6), a light chain CDR2 comprising GASSRAT (SEQ ID NO:7), and a light chain CDR3 comprising QQYSNFPI (SEQ ID NO:8).

61. The method of claim 49 or 50, wherein said anti-NOTCH antibody specifically binds human NOTCH2 and/or NOTCH3, wherein the antibody comprises:
(a) a heavy chain CDR1 comprising SSSGMS (SEQ ID NO:3), a heavy chain CDR2 comprising VIASSGSNTYYADSVKG (SEQ ID NO:4), and a heavy chain CDR3 comprising GIFFAI (SEQ ID NO:5); and
(b) a light chain CDR1 comprising RASQSVRSNYLA (SEQ ID NO:6), a light chain CDR2 comprising GASSRAT (SEQ ID NO:7), and a light chain CDR3 comprising QQYSNFPI (SEQ ID NO:8).

62. The method of claim 49 or 50, wherein said anti-NOTCH antibody specifically binds human NOTCH2 and/or NOTCH3, wherein the antibody comprises:
(a) a heavy chain variable region having at least about 90% sequence identity to SEQ ID NO:17, SEQ ID NO:18, or SEQ ID NO:26; and
(b) a light chain variable region having at least about 90% sequence identity to SEQ ID NO:29 or SEQ ID NO:27.

63. The method of claim 49 or 50, wherein said anti-NOTCH antibody comprises:
(a) a heavy chain variable region having at least about 95% sequence identity to SEQ ID NO:17; and
(b) a light chain variable region having at least about 95% sequence identity to SEQ ID NO:29.

64. The method of claim 49 or 50, wherein said anti-NOTCH antibody comprises:
(a) a heavy chain variable region having at least about 95% sequence identity to SEQ ID NO:18; and
(b) a light chain variable region having at least about 95% sequence identity to SEQ ID NO:29.

65. The method of claim 49 or 50, wherein said anti-NOTCH antibody comprises:
(a) a heavy chain variable region comprising SEQ ID NO:18; and
(b) a light chain variable region comprising SEQ ID NO:29.

66. The method of claim 49 or 50, wherein said anti-NOTCH antibody comprises:
(a) a heavy chain variable region comprising SEQ ID NO:17; and
(b) a light chain variable region comprising SEQ ID NO:29.

67. The method of claim 49 or 50, wherein said anti-NOTCH antibody competes for specific binding to human NOTCH2 and/or NOTCH3 with an antibody selected from the group consisting of:
(a) an antibody comprising a heavy chain variable region comprising SEQ ID NO:17 or SEQ ID NO:18, and a light chain variable region comprising SEQ ID NO:29;
(b) an antibody comprising a heavy chain CDR1 comprising SSSGMS (SEQ ID NO:3), a heavy chain CDR2 comprising VIASSGSNTYYADSVKG (SEQ ID NO:4), and a heavy chain CDR3 comprising SIFYTT (SEQ ID NO:9), and a light chain CDR1 comprising RASQSVRSNYLA (SEQ ID NO:6), a light chain CDR2 comprising GASSRAT (SEQ ID NO:7), and a light chain CDR3 comprising QQYSNFPI (SEQ ID NO:8); and
(c) an antibody encoded by the polynucleotide deposited with ATCC as PTA-9547.

68. The method of any one of claims 49-67, wherein said anti-NOTCH antibody is a chimeric antibody, a humanized antibody, a human antibody, or an antibody fragment.

69. The method of any one of claim 7, 23-28, or 47-68, further comprising administering a second therapeutic agent.

70. The method of claim 69, wherein the second therapeutic agent is a chemotherapeutic agent.

71. The method of claim 70, wherein the second therapeutic agent is a nucleoside analogue or a mitotic inhibitor.

72. The method of claim 69, wherein the second therapeutic agent is gemcitabine, paclitaxel, albumin-bound paclitaxel, or combinations thereof.

73. A diagnostic composition comprising an isolated polynucleotide comprising a sequence selected from the group consisting of SEQ ID NO: 35-43.

74. The diagnostic composition of claim 73, which comprises:
(a) a polynucleotide having the sequence of SEQ ID NO: 35, a polynucleotide having the sequence of SEQ ID NO: 36, and a polynucleotide having the sequence of SEQ ID NO: 37;
(b) a polynucleotide having the sequence of SEQ ID NO: 38, a polynucleotide having the sequence of SEQ ID NO: 39, and a polynucleotide having the sequence of SEQ ID NO: 40; or
(c) a polynucleotide having the sequence of SEQ ID NO: 41, a polynucleotide having the sequence of SEQ ID NO: 42, and a polynucleotide having the sequence of SEQ ID NO: 43.

75. A method of detecting NOTCH3 mRNA in a sample, comprising contacting the sample with a polynucleotide comprising a sequence selected from the group consisting of SEQ ID NO: 35-43.

76. The method of claim 75, which comprises contacting the sample with:
(a) a forward primer having the sequence of SEQ ID NO: 35, a reverse primer having the sequence of SEQ ID NO: 36, and a probe comprising an oligonucleotide having the sequence of SEQ ID NO: 37;
(b) a forward primer having the sequence of SEQ ID NO: 38, a reverse primer having the sequence of SEQ ID NO: 39, and a probe comprising an oligonucleotide having the sequence of SEQ ID NO: 40; or
(c) a forward primer having the sequence of SEQ ID NO: 41, a reverse primer having the sequence of SEQ ID NO: 42, and a probe comprising an oligonucleotide having the sequence of SEQ ID NO: 43.

77. A kit for detecting NOTCH3 mRNA in a sample, comprising a polynucleotide comprising a sequence selected from the group consisting of SEQ ID NO: 35-43.

78. The kit of claim 77, which comprises:
(a) a polynucleotide having the sequence of SEQ ID NO: 35, a polynucleotide having the sequence of SEQ ID NO: 36, and a polynucleotide having the sequence of SEQ ID NO: 37;
(b) a polynucleotide having the sequence of SEQ ID NO: 38, a polynucleotide having the sequence of SEQ ID NO: 39, and a polynucleotide having the sequence of SEQ ID NO: 40; or
(c) a polynucleotide having the sequence of SEQ ID NO: 41, a polynucleotide having the sequence of SEQ ID NO: 42, and a polynucleotide having the sequence of SEQ ID NO: 43.

79. A primer having a sequence selected from the group consisting of: SEQ ID NO: 35, SEQ ID NO: 36, SEQ ID NO: 38, SEQ ID NO: 39, SEQ ID NO: 41, and SEQ ID NO: 42.

80. A probe comprising an oligonucleotide having a sequence selected from the group consisting of SEQ ID NO: 37, SEQ ID NO: 40, and SEQ ID NO: 43.
